SEQUENCE LISTING (35 sequences)

SEQ ID NO: 1 - protein, 278 aa, artificial sequence
  chimeric polypeptide
MGRRRRRRRRRGGGPKKKRKVGGGGSTGEKPYKCPECGKSFSRSSNLQRHQRTHTGEKPYKCPECGKSFSRSDHLQTHQRTHTGEKPYKCPECGKSFSRSNHLQRHQRTHTGEKPYKCPECGKSFSRSDHLQRHQRTHTGEKPYKCPECGKSFSTSDHLQTHQRTHTGEKPYKCPECGKSFSRSNSLQRHQRTHGGGGSPRPTDVSLGDELHLDGEDVAMAHADALDDFDLDMLGDGDSPGPGFTPHDSAPYGALDMADFEFEQMFTDALGDYKDDDD

SEQ ID NO: 2 - protein, 279 aa, artificial sequence
  chimeric polypeptide
MGRRRRRRRRRGGGPKKKRKVGGGGSTGEKPYKCPECGKSFSRSTHLQRHQRTHTGEKPYKCPECGKSFSRSDHLQRHQRTHTGEKPYKCPECGKSFSRSDHLQRHQRTHTGEKPYKCPECGKSFSRSDSLQTHQRTHTGEKPYKCPECGKSFSTSNSLQRHQRTHTGEKPYKCPECGKSFSRSDHLQRHQRTHGGGGSPRPTDVSLGDELHLDGEDVAMAHADALDDFDLDMLGDGDSPGPGFTPHDSAPYGALDMADFEFEQMFTDALGDYKDDDDK

SEQ ID NO: 3 - DNA, 19 bp, artificial sequence
  synthetic oligonucleotide
gctcccaccccccacacac

SEQ ID NO: 4 - DNA, 19 bp, artificial sequence
  synthetic oligonucleotide
cgagggtggggggtgtgtg

SEQ ID NO: 5 - DNA, 19 bp, artificial sequence
  synthetic oligonucleotide
tccccccccccaaaacccc

SEQ ID NO: 6 - DNA, 19 bp, artificial sequence
  synthetic oligonucleotide
aggggggggggttttgggg

SEQ ID NO: 7 - protein, 7 aa, artificial sequence
  synthetic peptide
RSSNLQR

SEQ ID NO: 8 - protein, 7 aa, artificial sequence
  synthetic peptide
RSDHLQT

SEQ ID NO: 9 - protein, 7 aa, artificial sequence
  synthetic peptide
RSNHLQR

SEQ ID NO: 10 - protein, 7 aa, artificial sequence
  synthetic peptide
RSDHLQR

SEQ ID NO: 11 - protein, 7 aa, artificial sequence
  synthetic peptide
TSDHLQT

SEQ ID NO: 12 - protein, 7 aa, artificial sequence
  synthetic peptide
RSNSLQR

SEQ ID NO: 13 - protein, 7 aa, artificial sequence
  synthetic peptide
RSTHLQR

SEQ ID NO: 14 - protein, 7 aa, artificial sequence
  synthetic peptide
RSDHLQR

SEQ ID NO: 15 - protein, 7 aa, artificial sequence
  synthetic peptide
RSDHLQR

SEQ ID NO: 16 - protein, 7 aa, artificial sequence
  synthetic peptide
RSDSLQT

SEQ ID NO: 17 - protein, 7 aa, artificial sequence
  synthetic peptide
TSNSLQR

SEQ ID NO: 18 - protein, 7 aa, artificial sequence
  synthetic peptide
RSDHLQR

SEQ ID NO: 19 - protein, 28 aa, artificial sequence
  synthetic peptide
TGEKPYKCPECGKSFSRSSNLQRHQRTH

SEQ ID NO: 20 - protein, 28 aa, artificial sequence
  synthetic peptide
TGEKPYKCPECGKSFSRSDHLQTHQRTH

SEQ ID NO: 21 - protein, 28 aa, artificial sequence
  synthetic peptide
TGEKPYKCPECGKSFSRSNHLQRHQRTH

SEQ ID NO: 22 - protein, 28 aa, artificial sequence
  synthetic peptide
TGEKPYKCPECGKSFSRSDHLQRHQRTH

SEQ ID NO: 23 - protein, 28 aa, artificial sequence
  synthetic peptide
TGEKPYKCPECGKSFSTSDHLQTHQRTH

SEQ ID NO: 24 - protein, 28 aa, artificial sequence
  synthetic peptide
TGEKPYKCPECGKSFSRSNSLQRHQRTH

SEQ ID NO: 25 - DNA, 957 bp, Homo sapiens
taattttgtttaactttaagaaggagatataccatgggtcgtagacgcaggcgtagacgcaggcgtggtggcggtccgaagaaaaagcgtaaagtgggcggtggcggatccacgggtgagaagccgtataaatgtcccgaatgtggtaaaagttttagccgttcgacccatctgcaacgtcatcaacgcacccataccggcgaaaaaccatacaaatgtccggagtgcggcaaatctttctcgcgcagcgatcatttgcagcgccatcagagaactcacactggcgagaagccctacaagtgccccgaatgcgggaagagctttagtagatctgatcacttacaaaggcaccagaggacgcataccggtgagaagccgtataaatgtcccgaatgtggtaaaagttttagcacgtcggacagcctgcaaacccatcaacgcacccataccggcgaaaaaccatacaaatgtccggagtgcggcaaatctttctcgcgcagcaattcgttgcagcgtcatcagagaactcacactggcgagaagccctacaagtgccccgaatgcgggaagagctttagtagatctgatcatttacaacgccaccagaggacgcatacgggcgagaaaccgtatggtggcggtggcagccctaggccgaccgatgtgagcctgggcgatgaactgcatttagatggcgaagatgtggcgatggcccatgcggatgccctagacgattttgacctggatatgttaggcgatggtgacagccccggtccgggttttaccccgcatgatagcgcaccgtatggtgcgctagatatggcggatttcgaatttgaacagatgtttaccgatgcgctgggtattgatgaatatggcggtgctagcgattataaagatgacgatgacaaataagcggccgcactcgagcaccaccaccaccaccactgagatccg

SEQ ID NO: 26 - DNA, 957 bp, Homo sapiens
taattttgtttaactttaagaaggagatataccatgggtcgtagacgcaggcgtagacgcaggcgtggtggcggtccgaagaaaaagcgtaaagtgggcggtggcggatccacgggtgagaagccgtataaatgtcccgaatgtggtaaaagttttagccgttcgagcaatctgcaacgtcatcaacgcacccataccggcgaaaaaccatacaaatgtccggagtgcggcaaatctttctcgcgtagcgatcatttgcagacccatcagagaactcacactggcgagaagccctacaagtgccccgaatgcgggaagagctttagtagatctaatcacttacaacgccaccagaggacgcataccggtgagaagccgtataaatgtcccgaatgtggtaaaagttttagccgctcggaccatctgcaaagacatcaacgcacccataccggcgaaaaaccatacaaatgtccggagtgcggcaaatctttctcgaccagcgatcacttgcagacgcatcagagaactcacactggcgagaagccctacaagtgccccgaatgcgggaagagctttagtagatctaatagcttacaacgccaccagaggacgcatacgggcgagaaaccgtatggtggcggtggcagccctaggccgaccgatgtgagcctgggcgatgaactgcatttagatggcgaagatgtggcgatggcccatgcggatgccctagacgattttgacctggatatgttaggcgatggtgacagccccggtccgggttttaccccgcatgatagcgcaccgtatggtgcgctagatatggcggatttcgaatttgaacagatgtttaccgatgcgctgggtattgatgaatatggcggtgctagcgattataaagatgacgatgacaaataagcggccgcactcgagcaccaccaccaccaccactgagatccg

SEQ ID NO: 27 - DNA, 1340 bp, Homo sapiens
cccggagccggaccggggccaccgcgcccgctctgctccgacaccgcgccccctggacagccgccctctcctccaggcccgtggggctggccctgcaccgccgagcttcccgggatgagggcccccggtgtggtcacccggcgcgccccaggtcgctgagggaccccggccaggcgcggagatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatgaccaggtgtgtccacctgggcatatccaccacctccctcaccaacattgcttgtgccacaccctcccccgccactcctgaaccccgtcgaggggctctcagctcagcgccagcctgtcccatggacactccagtgccagcaatgacatctcaggggccagaggaactgtccagagagcaactctgagatctaaggatgtcacagggccaacttgagggcccagagcaggaagcattcagagagcagctttaaactcagggacagagccatgctgggaagacgcctgagctcactcggcaccctgcaaaatttgatgccaggacacgctttggaggcgatttacctgttttcgcacctaccatcagggacaggatgacctggagaacttaggtggcaagctgtgacttctccaggtctcacgggcatgggcactcccttggtggcaagagcccccttgacaccggggtggtgggaaccatgaagacaggatgggggctggcctctggctctcatggggtccaagttttgtgtattcttcaacctcattgacaagaactgaaaccaccaaaaaaaaaaaa

SEQ ID NO: 28 - protein, 193 aa, Homo sapiens
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 29 - protein, 7 aa, artificial sequence
  synthetic peptide
PKKKRKV

SEQ ID NO: 30 - protein, 5 aa, artificial sequence
  synthetic peptide
FDTDL

SEQ ID NO: 31 - protein, 23 aa, artificial sequence
  synthetic peptide
CXXXXCXXXFXXXXXLXXHXXXH

SEQ ID NO: 32 - protein, 4 aa, artificial sequence
  synthetic peptide
XXXX

SEQ ID NO: 33 - protein, 5 aa, artificial sequence
  synthetic peptide
PXXXX

SEQ ID NO: 34 - protein, 7 aa, artificial sequence
  synthetic peptide
PXXXXXX

SEQ ID NO: 35 - protein, 4 aa, artificial sequence
  synthetic peptide
XXXX

(19) United States
(12) Patent Application Publication
(10) Pub. No.: US 2010/0316702 A1
(43) Pub. Date: Dec. 16, 2010

(54) COMPOSITIONS AND METHODS FOR REGULATING ERYTHROPOEITIN EXPRESSION AND AMELIORATING ANEMIA AND STIMULATING ERYTHROPOIESIS

(75) Inventors: Steven P. Briggs, Del Mar, CA (US); Kiyoshi Tachikawa, San Diego, CA (US)

Correspondence Address: Joseph R. Baker, APC;Gavrilovich, Dodd & Lindsey LLP, 4660 La Jolla Village Drive, Suite 750, San Diego, CA, 92122, US

(73) Assignee: THE REGENTS OF THE UNIVERSITY OF CALIFORNIA, Oakland, CA (US)

(21) Appl. No.: 12/810,630

(22) PCT Filed: Jan. 8, 2009
(86) PCT No.: PCT/US09/30469

§ 371 Date: Aug. 26, 2010

Related U.S. Application Data
(60) Provisional application No. 61/019,815, filed on Jan. 8, 2008.

(51) Int. Cl. A61K 38/18; C07K 14/505; C07H 21/04; C12N 15/63; A61P 7/06; A61K 9/127; C12N 5/10

(52) U.S. Cl. 424/450; 530/380; 514/7.7; 536/23.1; 435/320.1; 435/325; 435/366; 435/363; 435/354; 435/353; 435/352; 435/350; 435/351

(57) ABSTRACT

The invention provides compositions (e.g., pharmaceuticals, formulations) and methods for ameliorating (e.g., preventing or treating) an anemia and/or stimulating erythropoiesis and/or EPO erythropoietin synthesis. The invention provides compositions comprising a chimeric protein artificial transcription factor comprising a plurality of (multiple) protein DNA-binding domains, e.g., zinc finger binding domains, specific for the promoter region of an erythropoietin (EPO) gene; a consensus nuclear localization protein sequence; a cell-penetrating peptide sequence; and a transcription activation domain.

### RELATED APPLICATIONS

This application claims the benefit of priority to U.S. Provisional Patent Application No. 61/019,815, filed Jan. 8, 2008. The aforementioned application is expressly incorporated herein by reference in its entirety and for all purposes.

### TECHNICAL FIELD

This invention relates to molecular and cellular biology, biochemistry, molecular genetics, protein therapy, and drug design and discovery. The invention provides compositions (e.g., pharmaceuticals, formulations) and methods for ameliorating (e.g., preventing or treating) anemia and/or stimulating erythropoiesis. The invention provides compositions comprising a chimeric protein artificial transcription factor comprising a plurality of (multiple) protein DNA-binding domains, e.g., zinc finger binding domains, specific for the promoter region of an erythropoietin (EPO) gene; a consensus nuclear localization protein sequence; a cell-penetrating peptide sequence; and, (4) a transcription activation domain.

### BACKGROUND

Erythropoietin (EPO, or hematopoietin or hemopoietin) is a glycoprotein hormone that is a cytokine for erythrocyte (red blood cell) precursors in the bone marrow. EPO is produced by the kidney. EPO is the hormone that regulates the red blood cell production (erythropoiesis), and lack of EPO can cause anemia.

### SUMMARY

The invention provides compositions and methods for ameliorating anemia and/or stimulating erythropoiesis and EPO synthesis. The invention provides chimeric proteins, or compositions comprising chimeric proteins, that act as artificial transcription factors, comprising:
(a) (1) a plurality of (multiple) EPO-specific DNA-binding domains (e.g., zinc finger DNA-binding domains) specific for the promoter region of (or any transcriptional regulatory region of) an erythropoietin (EPO) gene or a Erythropoietin Stimulating Protein (NESP) gene; (2) a consensus nuclear localization protein sequences; 3) a cell-penetrating peptide sequence; and, (4) a transcription activation domain;
(b) the chimeric protein of (a), wherein the chimeric protein comprises two, three, four, five, six or more EPO-specific DNA-binding domains (e.g., zinc finger DNA-binding domains) specific for an EPO gene;
(c) the chimeric protein of (a) or (b), wherein the EPO gene comprises a mammalian, e.g., a mouse and/or a human, EPO gene;
(d) the chimeric protein of any of (a) to (c), wherein the chimeric protein comprises one, two, three, four, five, six or more consensus nuclear localization protein sequences;
(e) the chimeric protein of any of (a) to (c), wherein the chimeric protein comprises one, two, three, four, five, six or more transcription activation domains; or
(f) the chimeric protein of any of (a) to (e), wherein the chimeric protein comprises a formulation for subcutaneous, parenteral, topical, oral or local administration, or for aerosol or transdermal administration.

The invention provides recombinant or synthetic nucleic acids comprising nucleic acid sequence encoding a chimeric protein of this invention.

The invention provides vectors, plasmids, recombinant viruses or expression vehicles comprising a nucleic acid of this invention.

The invention provides pharmaceutical formulations comprising
(a) a chimeric protein of this invention; a nucleic acid of this invention; and/or the vector, plasmid, recombinant virus or expression vehicle of this invention; or,
(b) the pharmaceutical formulation of (a) formulated for subcutaneous, parenteral, topical, oral or local administration, or for aerosol or transdermal administration.

The invention provides methods for ameliorating an anemia in an individual comprising:
(a) (i) a pharmaceutical formulation of this invention; a chimeric protein of this invention; a nucleic acid of this invention; and/or a vector, plasmid, recombinant virus or expression vehicle of this invention; and
(ii) administering an effective amount of the composition of (a) to an individual in need thereof;
(b) the method of (a), wherein the individual is a mammal (e.g., a mouse, a human);
(c) the method of (a) or (b), wherein the composition comprises a pharmaceutical formulation;
(d) the method of (c), wherein the pharmaceutical formulation comprises a formulation for subcutaneous, parenteral, topical, oral or local administration, or for aerosol or transdermal administration; or
(e) the method of any of (a) to (d), wherein the anemia is caused by cancer, or a cancer therapeutic or therapy.

The invention provides kits comprising (a) (i) the chimeric protein of this invention; the nucleic acid of this invention; and/or the vector, plasmid, recombinant virus or expression vehicle of this invention; and, (b) instructions to perform the method of this invention. Also provided are kits comprising instructions for practicing the methods of the invention.

The invention provides chimeric proteins comprising:
(a) (1) a plurality of (multiple) DNA-binding domains specific for (that can specifically bind to) a promoter and/or another transcriptional regulatory region of an erythropoietin (EPO) gene or a Erythropoietin Stimulating Protein (NESP) gene;
(2) at least one nuclear localization peptide (NLP) domain;
(3) at least one cell-penetrating peptide (CPP); and,
(4) at least one transcription activation (TA) domain; or
(b) the chimeric protein of (a), comprising an EPO-specific zinc finger DNA-binding domain, or an NESP-specific zinc finger DNA-binding domain.

In alternative embodiments, the chimeric protein, the EPO- or NESP-specific DNA-binding domain, the NLP domain, the CPP domain and/or the TA domain comprises or consist of a recombinant protein, a synthetic protein, a peptidomimetic, a non-natural peptide or a combination thereof.

In alternative embodiments, the EPO or NESP gene or transcriptional regulatory region of an EPO or NESP gene comprises (or consists of): (a) a mammalian EPO or NESP gene or transcriptional regulatory region or (b) a mouse or a human EPO or NESP gene or transcriptional regulatory region. The transcriptional regulatory region can comprise (or consist of) a promoter or an enhancer, an EPO or NESP promoter or EPO or NESP enhancer, or a synthetic promoter. The chimeric protein can comprise (or consist of) multiple copies of the EPO- or NESP-specific DNA-binding domain, the NLP, the CPP and/or the TA domain. In alternative embodiments, the chimeric protein comprises (or consists of) two, three, four, five, six or more EPO- or NESP-specific DNA-binding domains specific for (that can specifically bind to) a promoter and/or another transcriptional regulatory region of an EPO or NESP gene. The chimeric protein can comprises (or consists of) one, two, three, four, five, six or more nuclear localization peptide (NLP) domains or consensus nuclear localization proteins.

In alternative embodiments: the chimeric protein comprises one, two, three, four, five, six or more cell-penetrating peptides (CPPs); or, the chimeric protein comprises one, two, three, four, five, six or more TA domains, and/or one or more other functional domains with a histone acetyltransferase (HAT) activity. The at least one TA domain can comprise a herpes simplex virus (HSV) VP-16 activation peptide domain or a peptide derived from the C-terminal transcription activation domain of β-catenin (FDTDL). The at least one (the one or more) zinc finger DNA-binding domain(s) can comprise: (1) a zinc-finger of the C₂H₂ class; (2) a zinc-finger of the C₄ class; or (3) a zinc-finger of C₆ class. The at least one (the one or more) zinc finger DNA-binding domain(s) can comprise (or consist of) the consensus sequence Cys-X₂₋₄-Cys-X₃-Phe-X₅-Leu-X₂-His-X3-His.

In alternative embodiments: the at least one nuclear localization peptide (NLP) domain comprises: (1) an NLP sequence of a large T antigen of the simian virus 40 (SV-40), or PKKKRKV (SEQ ID NO:2); (2) a consensus sequence fitting B₄, P(B₃X), PXX(B₃X), B₃(H/P), where B is a basic amino acid, P is proline, H is histidine, X is any amino acid and letters in parentheses can be in any order; (3) a bipartite NLP comprising two short stretches of basic amino acids separated by a non-conserved sequence; or (4) a cellular nucleoplasmin protein KRPAATKKAGQAKKKK (SEQ ID NO:4).

In alternative embodiments: the at least one cell-penetrating peptide (CPP) comprises: (1) a plurality of polycationic amino acid residues; (2) a plurality of arginine amino acid residues; or (3) a TAT protein (Trans-acting Activator of Transcription) of a Human Immunodeficiency Virus (HIV-1).

In alternative embodiments: (1) the at least one TA domain is at least approximately 25% hydrophobic and is linked to the at least one zinc finger DNA-binding domain in a manner that does not interfere with the promoter or a transcriptional regulatory binding activity of the zinc finger DNA binding peptide, and the TA domain is both necessary and sufficient to activate transcription of the gene; and/or (2) the TA domain is between about 5 to 25 amino acids in length, or is between about 6 to 20 amino acids in length, or is about 5, 6, 7, 8, 9, 10, 11, 11, 12, 13, 14 or 15 amino acids in length.

In alternative embodiments: the at least one TA domain comprises a herpes simplex virus (HSV) VP-16 activation peptide domain or a peptide derived from the C-terminal transcription activation domain of β-catenin (FDTDL).

In alternative embodiments: at least one, or all, of the domains and/or chimeric proteins further comprises, or is attached to, a lipid, a hydrophobic alkane or alkene (olefin) moiety, or a polyethylene glycol (PEG) moiety. The at least one, several, or all, of the chimeric proteins can further comprises, or be attached to, an epitope peptide tag or a detectable composition or moiety. The detectable composition or moiety can comprise a phosphoprotein, a fluorescent molecule, a fluorescent tagged protein, a radiolabel or a radiolabeled protein.

In alternative embodiments chimeric proteins of the invention can further comprise a small molecule, a hormone or a cytokine that increases or upregulates erythropoiesis or red blood cell production in a mammalian.

In alternative embodiments chimeric proteins of the invention can comprise (or be formulated or made as): (a) a formulation for subcutaneous, parenteral, topical, oral or local administration, or for aerosol or transdermal administration, or administration by nebulizer; or (b) the chimeric protein of (a), wherein the topical formulation comprises an ointment, a cream, a powder, an emulsion, a gel, a glycerogelatin, a paste, a plaster, a sprayable composition or a lotion.

The invention provides compositions comprising a plurality of one or more chimeric proteins of the invention, or a formulation of the invention. The composition can further comprise a small molecule, a hormone or a cytokine that increases or upregulates erythropoiesis or red blood cell production in a mammalian. The composition can further comprise a synthetic or a recombinant erythropoietin, e.g., a human synthetic or a recombinant erythropoietin.

The invention provides liquids, gels, hydrogels, powders or aqueous formulations comprising one or more chimeric proteins of the invention, or a composition or formulation of the invention.

The invention provides vesicles, liposomes, nanoparticles or nanolipid particles (NLP) comprising one or more chimeric proteins of the invention, or a composition or formulation of the invention, or a liquid, gel, hydrogel, powder or aqueous formulation of the invention.

The invention provides cells, e.g., isolated, host or cultured cells, comprising (or having contained therein) one or more chimeric proteins of the invention (or a nucleic acid, such as a vector, encoding a chimeric protein of the invention), or a composition or formulation of the invention, or a liquid, gel, hydrogel, powder or aqueous formulation of the invention. These cells, e.g., isolated, host or cultured cells, can be mammalian cells, such as human cells, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell. These cells, e.g., isolated, host or cultured cells, also can be prokaryotic cells, yeast cells, fungal cells, insect cells or plant cells.

The invention provides formulations, e.g., pharmaceutical or sterile formulations, comprising one or more chimeric proteins of the invention (or a nucleic acid, such as a vector, encoding a chimeric protein of the invention), or a composition or formulation of the invention, or a liquid, gel, hydrogel, powder or aqueous formulation of the invention, a vesicle, liposome, nanoparticle or nanolipid particle (NLP) of the invention, or an isolated or cultured cell of the invention.

The invention provides products of manufacture comprising one or more chimeric proteins of the invention (or a nucleic acid, such as a vector, encoding a chimeric protein of the invention), or a composition or formulation of the invention, or a liquid, gel, hydrogel, powder or aqueous formulation of the invention, a vesicle, liposome, nanoparticle or nanolipid particle (NLP) of the invention, or an isolated or cultured cell of the invention.

The invention provides recombinant or synthetic nucleic acids (e.g., including vectors, plasmids, recombinant viruses or expression vehicles) comprising a nucleic acid sequence encoding a chimeric protein of the invention. The invention provides vectors, plasmids, recombinant viruses and expression vehicles comprising: (a) a nucleic acid of the invention; (b) the vector, plasmid, recombinant virus or expression vehicle of (a), wherein the nucleic acid is operatively linked to a constitutive promoter or an inducible promoter; or (c) the vector, plasmid, recombinant virus or expression vehicle of (a) or (b), wherein the promoter is only active in a hematopoietic cell.

The invention provides cells, e.g., isolated, host or cultured cells, comprising (or having contained therein) a nucleic acid of the invention, or a vector, plasmid, recombinant virus or expression vehicle of the invention. These cells, e.g., isolated or cultured cells, can be mammalian cells, such as human cells, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell. These cells, e.g., isolated or cultured cells, also can be prokaryotic cells, yeast cells, fungal cells, insect cells or plant cells.

The invention provides formulations, including pharmaceutical or sterile formulations, comprising:
(a) (i) a chimeric protein of the invention, a liquid, gel, hydrogel, powder or aqueous formulation of the invention, a vesicle, liposome, nanoparticle or nanolipid particle (NLP) of the invention, or an isolated, host or cultured cell of the invention, and (ii) a pharmaceutically acceptable excipient;
(b) the pharmaceutical formulation or sterile formulation of (a) formulated for subcutaneous, parenteral, topical, oral or local administration, or for aerosol or transdermal administration, or administration by a nebulizer; or
(c) the pharmaceutical formulation or sterile formulation of (a) is formulated for topical application in the form of an ointment, a cream, a powder, an emulsion, a gel, a glycerogelatin, a paste, a plaster, a sprayable composition or a lotion.

The invention provides methods for ameliorating or preventing an anemia, and/or stimulating erythropoiesis and/or erythropoietin (EPO) synthesis, in an individual comprising: (a) providing: a pharmaceutical or sterile formulation of the invention; a pharmaceutical formulation of the invention; a chimeric protein of the invention; a liquid, gel, hydrogel, powder or aqueous formulation of the invention; a vesicle, liposome, nanoparticle or nanolipid particle (NLP) of the invention; an isolated, host or cultured cell of the invention; a nucleic acid of the invention; or a vector, plasmid, recombinant virus or expression vehicle of the invention; and (b) administering an effective amount of (a) to an individual in need thereof. The individual in need thereof can be a mammalian or a human. The anemia ameliorated or prevented can be caused by a genetic disorder, an infection, a dietary disorder or deficiency, a pollutant, a pesticide, herbicide or insecticide, a poison, a venom, a toxin, a biological agent, a drug, a cancer or a cancer therapeutic or cancer therapy. In one aspect of the method, the anemia ameliorated or prevented is a microcytic, normocytic or macrocytic form of anemia. The anemia ameliorated or prevented can be: a drug-induced anemia; caused by an infection; caused by an iron deficiency; caused by rhesus disease (hemolytic disease of newborn); caused by sickle-cell disease, thalassemia or Plummer-Vinson syndrome (PVS, also called Paterson-Brown-Kelly syndrome or sideropenic dysphagia); a sideroblastic anemia-congenital or acquired; caused by Gaucher's disease; caused by a vitamin deficiency; caused by autoimmune hemolytic anemia (AIHA); caused by a cancer; or, caused by heavy metal poisoning or pyridoxine deficiency. The vitamin deficiency can be a folate or B12 deficiency (pernicious anemia or Addison's anemia). The drug-induced anemia can be caused by methyldopa or fludarabin. The AIHA can be caused by Systemic lupus erythematosus, a drug, Evans syndrome, chronic lymphocytic leukemia or is idiopathic. The cancer can be chronic lymphocytic leukemia, small cell lymphoma (or small lymphocytic lymphoma) or a non-Hodgkin's lymphoma; or the anemia is caused by myelophythisis secondary to an acute megakaryoblastic leukemia, a lymphoma, a myeloma or a carcinoma metastatic to bone marrow. In alternative embodiments of methods of the invention, the infection is an EBV infectious mononucleosis, a Babesiosis infection, or equine infectious anemia. The cancer therapeutic or cancer therapy can be radiotherapy, hormone therapy or chemotherapy. The heavy metal poisoning can be lead poisoning, mercury poisoning (hydrargaria), copper poisoning, nickel poisoning, manganese poisoning (manganism) or cadmium poisoning.

The invention provides kits comprising a pharmaceutical or sterile formulation of the invention; a pharmaceutical formulation of the invention; a chimeric protein of the invention; a liquid, gel, hydrogel, powder or aqueous formulation of the invention; a vesicle, liposome, nanoparticle or nanolipid particle (NLP) of the invention; an isolated, host or cultured cell of the invention; a nucleic acid of the invention; or a vector, plasmid, recombinant virus or expression vehicle of the invention.

The invention provides uses of a pharmaceutical or sterile formulation of the invention; a chimeric protein of the invention; a liquid, gel, hydrogel, powder or aqueous formulation of the invention; a vesicle, liposome, nanoparticle or nanolipid particle (NLP) of the invention; an isolated, host or cultured cell of the invention; a nucleic acid of the invention; or a vector, plasmid, recombinant virus or expression vehicle of the invention, to make a pharmaceutical composition for ameliorating or preventing an anemia in an individual. In alternative embodiments, the individual in need thereof is a mammal or a human. The anemia ameliorated or prevented can be caused by a genetic disorder, an infection, a dietary disorder or deficiency, a pollutant, a pesticide, herbicide or insecticide, a poison, a venom, a toxin, a biological agent, a drug, a cancer or a cancer therapeutic or cancer therapy. The anemia ameliorated or prevented can be a microcytic, normocytic or macrocytic form of anemia. The anemia ameliorated or prevented can be: a drug-induced anemia; caused by an infection; caused by an iron deficiency; caused by rhesus disease (hemolytic disease of newborn); caused by sickle-cell disease, thalassemia or Plummer-Vinson syndrome (PVS, also called Paterson-Brown-Kelly syndrome or sideropenic dysphagia); a sideroblastic anemia-congenital or acquired; caused by Gaucher's disease; caused by a vitamin deficiency; caused by autoimmune hemolytic anemia (AIHA); caused by a cancer; or, caused by heavy metal poisoning or pyridoxine deficiency. The vitamin deficiency can be a folate or B12 deficiency (pernicious anemia or Addison's anemia). The drug-induced anemia can be caused by methyldopa or fludarabin. The AIHA can be caused by Systemic lupus erythematosus, a drug, Evans syndrome, chronic lymphocytic leukemia or is idiopathic. The cancer can be chronic lymphocytic leukemia, small cell lymphoma (or small lymphocytic lymphoma) or a non-Hodgkin's lymphoma; or the anemia is caused by myelophythisis secondary to an acute megakaryoblastic leukemia, a lymphoma, a myeloma or a carcinoma metastatic to bone marrow. The infection can be an EBV infectious mononucleosis, a Babesiosis infection, or equine infectious anemia. The cancer therapeutic or cancer therapy can be radiotherapy, hormone therapy or chemotherapy. The heavy metal poisoning can be lead poisoning, mercury poisoning (hydrargaria), copper poisoning, nickel poisoning, manganese poisoning (manganism) or cadmium poisoning.

The invention provides methods for upregulating or activating an erythropoietin (EPO) gene in a cell comprising: (a) providing: a pharmaceutical or sterile formulation of the invention; a chimeric protein of the invention; a liquid, gel, hydrogel, powder or aqueous formulation of the invention; a vesicle, liposome, nanoparticle or nanolipid particle (NLP) of the invention; a isolated, host or cultured cell of the invention; a nucleic acid of the invention; a product of manufacture of the invention; or a vector, plasmid, recombinant virus or expression vehicle of the invention; and (b) contacting or administering an effective amount of (a) to the cell, thereby upregulating or activating an erythropoiesis gene in a cell. The cell can be a mammalian cell, e.g. such as a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell. In alternative embodiments, the contacting or administering is in vitro, ex vivo or in vivo.

The invention provides methods for making a recombinant erythropoietin (EPO) in a cell comprising: (a) providing: a pharmaceutical or sterile formulation of the invention; a chimeric protein of the invention; a liquid, gel, hydrogel, powder or aqueous formulation of the invention; a vesicle, liposome, nanoparticle or nanolipid particle (NLP) of the invention; a isolated, host or cultured cell of the invention; a nucleic acid of the invention; a product of manufacture of the invention; or a vector, plasmid, recombinant virus or expression vehicle of the invention; (b) inserting in a cell a nucleic acid comprising a heterologous EPO coding sequence operatively linked to an EPO specific promoter; and (c) contacting or administering an effective amount of (a) to the cell, thereby making a recombinant EPO in the cell. The cell can be a mammalian cell, e.g., such as a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell. The contacting or administering can be in vitro, ex vivo or in vivo.

The invention provides methods for increasing hematocrit in a mammal comprising: (a) providing: a pharmaceutical or sterile formulation of the invention; a chimeric protein of the invention; a liquid, gel, hydrogel, powder or aqueous formulation of the invention; a vesicle, liposome, nanoparticle or nanolipid particle (NLP) of the invention; a isolated, host or cultured cell of the invention; a nucleic acid of the invention; a product of manufacture of the invention; or a vector, plasmid, recombinant virus or expression vehicle of the invention; and (b) contacting or administering an effective amount of (a) to the mammal, thereby increasing the mammal's hematocrit. The cell can be a mammalian cell, e.g., such as a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell. The contacting or administering can be in vitro, ex vivo or in vivo.

The invention provides methods for increasing endogenous erythropoietin (EPO) production in an individual or cell in need thereof, the method comprising: (a) providing: a pharmaceutical or sterile formulation of the invention; a chimeric protein of the invention; a liquid, gel, hydrogel, powder or aqueous formulation of the invention; a vesicle, liposome, nanoparticle or nanolipid particle (NLP) of the invention; a isolated, host or cultured cell of the invention; a nucleic acid of the invention; a product of manufacture of the invention; or a vector, plasmid, recombinant virus or expression vehicle of the invention; and (b) contacting or administering an effective amount of (a) to the individual, thereby increasing endogenous erythropoietin (EPO) production. The cell can be a mammalian cell, e.g., such as a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell. The contacting or administering can be in vitro, ex vivo or in vivo.

In alternative embodiments, the individual can be a human or an animal. The effective amount of (a) can be administered to the individual to treat, ameliorate and/or prevent: myelodysplastic syndrome; a peripheral nerve injury; a hemoglobin H disease; a testicular torsion-detorsion; post-infarct myocardial damage; or a neurologic disorder; and/or, to treat or ameliorate a wound and/or an inflammation, or to treat or ameliorate conjunctivitis; surgical or accidental wounds; a bedsore; a burn; an inflammation of the skin, mucous membranes, airways or lungs; an eczema or a skin disorder accompanied by necrosis, by dermatitis, by psoriasis or by diabetes mellitus. The effective amount of (a) can be administered as a neuroprotective in low birth weight infants. The effective amount of (a) can be administered as a neuroprotective for altitude related illnesses. The neurologic disorder can be ischemic stroke, intracerebral hemorrhage, subarachnoid hemorrhage, traumatic brain injury or Parkinson's disease.

The invention provides use of a pharmaceutical or sterile formulation of the invention; a chimeric protein of the invention; a liquid, gel, hydrogel, powder or aqueous formulation of the invention; a vesicle, liposome, nanoparticle or nanolipid particle (NLP) of the invention; a isolated, host or cultured cell of the invention; a nucleic acid of the invention; a product of manufacture of the invention; or a vector, plasmid, recombinant virus or expression vehicle of the invention, to make a pharmaceutical composition to: (a) treat, ameliorate and/or prevent: a myelodysplastic syndrome; a peripheral nerve injury; a hemoglobin H disease; a testicular torsion-detorsion; post-infarct myocardial damage; or a neurologic disorder; (b) act as a neuroprotective in low birth weight infants; (c) as a neuroprotective for altitude related illnesses; or (d) to treat or ameliorate a wound and/or an inflammation, or to treat or ameliorate conjunctivitis; surgical or accidental wounds; a bedsore; a burn; an inflammation of the skin, mucous membranes, airways or lungs; an eczema or a skin disorder accompanied by necrosis, by dermatitis, by psoriasis or by diabetes mellitus. The neurologic disorder can be ischemic stroke, intracerebral hemorrhage, subarachnoid hemorrhage, traumatic brain injury or Parkinson's disease.

The details of one or more embodiments of the invention are set forth in the accompanying drawings and the description below. Other features, objects, and advantages of the invention will be apparent from the description and drawings, and from the claims. All publications, patents, patent applications cited herein are hereby expressly incorporated by reference for all purposes.

### BRIEF DESCRIPTION OF THE DRAWINGS

The following drawings are illustrative of embodiments of the invention and are not meant to limit the scope of the invention as encompassed by the claims.

FIG. 1 illustrates photomicrograph images that confirms that chimeric proteins (also called “Designed Regulatory Proteins”, or DRPs) of this invention can enter mammalian cells, using in this example human lymphocyte cells: left two panels illustrate a fluorescence image, and right two panels illustrate a phase contrast image; the upper two panels illustrate detection of green fluorescent protein expressed by chimeric proteins (DRPs) of the invention, as discussed in detail in Example 1, below.

FIG. 2 graphically illustrates data from an ELISA assay demonstrating that chimeric proteins (DRPs) of this invention (the exemplary EPO1-DRP and EPO2-DRP) when inserted into mammalian cells can enhance secretion of EPO protein in those cells, as discussed in detail in Example 1, below.

FIG. 3 graphically illustrates data demonstrating blood EPO levels in mice after administration of exemplary chimeric proteins (DRPs) of this invention (EPO1-DRP and EPO2-DRP); as discussed in detail in Example 1, below.

FIG. 4 schematically illustrates exemplary target DNA sequences for exemplary chimeric proteins (DRPs) of this invention, particularly, the zinc finger DNA-binding domains of chimeric proteins (DRPs) of this invention; as discussed in detail in Example 2, below.

FIG. 5 illustrates alternative exemplary zinc finger DNA target sequences for chimeric proteins (DRPs) of the invention, with the corresponding exemplary zinc finger DNA-binding motif; as discussed in detail in Example 2, below.

FIG. 6 illustrates exemplary chimeric proteins comprising one or more or all of the listed amino acid zinc finger DNA-binding motifs; as discussed in detail in Example 2, below.

Like reference symbols in the various drawings indicate like elements.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

The invention provides compositions and methods for the prevention or amelioration, including the treatment of, anemia and/or stimulating erythropoiesis and EPO synthesis. The invention provides compositions and methods for the amelioration or prevention of anemia caused by any disease or condition, including anemic phenotypes caused by any genotype, e.g., any genetic predisposition, such as a mutation in a gene necessary to maintain normal erythropoiesis and/or red blood cell counts. However, in alternative embodiments the anemias ameliorated or prevented are not necessarily caused by a particular genotype, but rather can be caused by exposure to an environmental agent, e.g., a drug, food, insecticide, herbicide, pesticide, poison, venoms, toxin, biological warfare agent, pollutant, allergen and the like. In some embodiments, a combination of these elements cause the anemia, e.g., one or more environmental agents and a genetic predisposition. In alternative embodiments the anemias ameliorated or prevented are caused by a disease or condition, e.g., cancer, folate or B12 deficiency, autoimmune hemolytic anemia and the like.

The invention provides compositions and methods for a protein therapeutic-based approach for manipulating, e.g., stimulating or inhibiting, the expressing of erythropoietin (EPO) coding sequences, including EPO genes. In one embodiment, compositions and methods of the invention are used to stimulate an endogenous and/or exogenous or heterologous mammalian, e.g., human, erythropoietin (EPO) gene, where the EPO gene is stimulated by compositions comprising chimeric proteins (DRPs) of the invention. For example, in one embodiment, a chimeric protein (DRP) of the invention is used ex vivo or in vivo to stimulate an exogenous or heterologous mammalian EPO gene inserted into a cell, including e.g. a transgenic animal comprising an exogenous or heterologous EPO gene, or cells with exogenous or heterologous mammalian EPO genes implanted in an animal or individual.

In alternative embodiments, chimeric proteins (DRPs) of this invention comprise (or consist of) an artificial transcription factor comprising (or consisting of) (1) one or multiple (two, three, four, five, six or more) EPO-specific DNA-binding domains (e.g., zinc finger DNA-binding domains) that can bind to an EPO gene transcriptional regulatory sequence and/or an Erythropoietin Stimulating Protein (NESP) gene transcriptional regulatory sequence; such as a promoter and/or an enhancer, and in one embodiment, the zinc finger DNA-binding domain is specific for an EPO gene and/or an Erythropoietin Stimulating

Protein (NESP) gene; 2) one or multiple consensus nuclear localization sequences; 3) one or multiple cell-penetrating peptide sequences and 4) one or multiple transcription activation (TA) domains.

Alternative embodiments include nucleic acids that encode the chimeric proteins (DRPs) of this invention, including expression cassettes, plasmids, vectors and the like comprising these DPR-coding sequences. Alternative embodiments include host cells, e.g., cultured, isolated or transformed cells, comprising nucleic acids and/or chimeric proteins of this invention.

In alternative embodiments, the erythropoietin (EPO) coding sequence (e.g., a gene) activated by a chimeric protein (DRP) of this invention is endogenous and/or exogenous or heterologous; for example, a cell can be supplemented with an exogenous or heterologous EPO coding sequence (e.g., a gene) and administration of a chimeric protein of the invention or a nucleic acid encoding a chimeric protein of the invention activates the exogenous or heterologous EPO coding sequence, the endogenous EPO coding sequence or both.

In one aspect of the invention, compositions comprise EPO gene-specific chimeric proteins (e.g., the chimeric proteins of the invention can target transcriptional activators such as promoter of EPO genes). In one embodiment, these compositions of the invention are used as a therapy for humans and other mammals with anemia (e.g., to treat or ameliorate anemia), or to prevent anemia. In one embodiment, after therapy with an EPO-specific DRP-comprising composition of this invention (e.g., a pharmaceutical composition), EPO gene transcription occurs (EPO coding sequence is generated). In one embodiment, EPO protein is secreted from an individual's, e.g., a patient, a human, any mammal. In one embodiment, EPO protein is secreted from cells and enters the individual's bloodstream in clinically significant amounts. In one embodiment, EPO protein is secreted from the individual's intestinal cells and enters the bloodstream in clinically significant amounts. In one embodiment, chimeric proteins of this invention, including e.g., compositions comprising these DRPs, are used for mammalian, e.g., human, erythropoietin (EPO) manipulation, e.g., activation or upregulation, and can include pharmaceutical preparations (formulations) to activate the endogenous and/or an exogenous EPO gene in vitro, ex vivo and/or in vivo.

In one embodiment, the invention comprises selecting a target site in the EPO gene, e.g., an EPO promoter or other transcriptional regulatory sequence, that can be specifically bound by a DNA-binding domain, e.g., a zinc finger DNA-binding domain, and then designing and manufacturing a chimeric (e.g., a recombinant) protein that will bind that EPO DNA sequence with high affinity and specificity.

In one embodiment, the invention provides a chimeric (e.g., a recombinant) protein with the ability to enter cells (e.g., human, mammalian or other cells), go to the nucleus, bind specifically to the EPO gene (or equivalent), and activate transcription of the EPO gene (or equivalent), resulting in accumulation of EPO (or equivalent) in the cell and/or secretion of EPO (or equivalent) from the cell, and alternatively, secretion or release of EPO (or equivalent) into the bloodstream.

In one embodiment, chimeric proteins of this invention comprise a plurality (e.g., two, three, four or more) domains that enable each of at least four activities: at least one (one or more) protein transduction domain(s); at least one (one or more) nuclear localization signal domain(s); at least one (one or more) EPO specific DNA-binding domain(s) (e.g., comprising one, two, three, four, five, six or more zinc fingers), and at least one (one or more) transactivation domain(s).

### Polypeptides and Peptides

The invention provides chimeric polypeptides for ameliorating (e.g., preventing or treating) anemia and/or stimulating erythropoiesis and EPO synthesis; and in alternative embodiments chimeric proteins of the invention comprise: one or more (a plurality of, multiple) protein DNA-binding domains, e.g., zinc finger binding domains, specific for the promoter region of an erythropoietin (EPO) gene or an Erythropoietin Stimulating Protein (NESP); one or more consensus nuclear localization proteins; one or more cell-penetrating peptides; and, one or more transcription activation domain. Erythropoietin (EPO) and Erythropoietin Stimulating Protein (NESP) polypeptides and genes encoding them are well known in the art. For example, see U.S. Pat. Nos. 7,262,166, and 6,586,398, describing the Erythropoietin Stimulating Protein (NESP) protein and gene.

In alternative embodiments, polypeptides and/or peptides used to practice the invention comprise a recombinant protein, a synthetic protein, a peptidomimetic, a non-natural peptide, or a combination thereof. Peptides and proteins used to practice the invention can be recombinantly expressed in vitro or in vivo. The peptides and polypeptides used to practice the invention can be made and isolated using any method known in the art. Polypeptide and peptides used to practice the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) including any automated polypeptide synthesis process known in the art.

The chimeric peptides and polypeptides used to practice the invention can also be glycosylated. The glycosylation can be added post-translationally either chemically or by cellular biosynthetic mechanisms, wherein the later incorporates the use of known glycosylation motifs, which can be native to the sequence or can be added as a peptide or added in the nucleic acid coding sequence. The glycosylation can be O-linked or N-linked.

In alternative embodiments, compositions of the invention, including the chimeric peptides and polypeptides used to practice the invention, can comprise an oligopeptide, peptide, polypeptide, or protein sequence, or to a fragment, portion, or subunit of any of these and to naturally occurring or synthetic molecules, including, e.g., peptidomimetics and non-natural amino acids. In alternative embodiments, chimeric peptides and polypeptides used to practice the invention comprise amino acids joined to each other by peptide bonds or modified peptide bonds and may comprise modified amino acids other than the 20 gene-encoded amino acids. The chimeric peptides and polypeptides may be modified by either natural processes, such as post-translational processing, or by chemical modification techniques that are well known in the art. Modifications can be designed anywhere in the polypeptide, including the peptide backbone, the amino acid side-chains and the amino or carboxyl termini. The same type of modification can be made in the same or varying degrees at several sites in a given chimeric polypeptide.

In alternative embodiments, chimeric peptides and polypeptides used to practice the invention can have many types of modifications, e.g., modifications including acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, phosphorylation, prenylation, racemization, selenoylation, sulfation and transfer-RNA mediated addition of amino acids to protein such as arginylation. See for example, Creighton, T. E., Proteins—Structure and Molecular Properties 2nd Ed., W.H. Freeman and Company, New York (1993); Posttranslational Covalent Modification of Proteins, B. C. Johnson, Ed., Academic Press, New York, pp. 1-12 (1983)). In another embodiment, a chimeric polypeptide of the invention (a DRP) can be glycol-pegylated as described in U.S. Pat. No. 7,405,198; or can be glycosylated as described in U.S. Pat. Nos. 7,276,475 or 7,399,613, or 7,338,933, the later describing O-linked glycosylation of peptides. In one embodiment, a chimeric polypeptide of the invention (a DRP) used to practice this invention can be acylated as described e.g., in U.S. Pat. No. 7,273,921. In another embodiment, a chimeric polypeptide of the invention (a DRP) can be pegylated (a process that can be called “PEGylation”) as described e.g., in U.S. Patent App. Pub. No. 20030166566 and 20060264377.

In alternative embodiments, chimeric peptides and polypeptides used to practice the invention can comprise any “mimetic” and/or “peptidomimetic” form. In alternative embodiments, peptides and polypeptides used to practice the invention can comprise synthetic chemical compounds which have substantially the same structural and/or functional characteristics of natural polypeptides. A mimetic used to practice the invention can be either entirely composed of synthetic, non-natural analogues of amino acids, or, is a chimeric molecule of partly natural peptide amino acids and partly non-natural analogs of amino acids. A mimetic used to practice the invention can also incorporate any amount of natural amino acid conservative substitutions as long as such substitutions also do not substantially alter the mimetic's structure and/or activity.

Routine experimentation will determine whether a synthetic molecule or mimetic is effective for practicing the invention, e.g., has EPO promoter-specific binding activity, e.g., an EPO-specific zinc finger DNA-binding activity, or a nuclear localization peptide activity, or a cell-penetrating peptide activity, or a transcription activation (TA) peptide domain and/or a transcription repression peptide activity. Methodologies detailed herein and others known to persons skilled in the art may be used to select or guide one to choose effective mimetic for practicing the compositions and/or methods of this invention.

Polypeptide mimetic compositions for practicing the invention can comprise any combination of non-natural structural components. In alternative aspects, mimetic compositions for practicing the invention can comprise one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and

Biochemistry of Amino Acids, Peptides and Proteins, Vol. 7, pp 267-357, “Peptide Backbone Modifications,” Marcell Dekker, NY). A polypeptide can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; D- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids used to practice this invention can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholinyl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions. Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, e.g., under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics that can be used include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

Polypeptides used to practice this invention can comprise any heterologous sequence, e.g., a “tag” for identifying and/or isolating a chimeric polypeptide of the invention, and/or a signal sequence (a leader sequence) e.g., for secreting a recombinant antibody or inhibitory polypeptide used to practice the invention from a cell, e.g., a production host cell.

### Generating and Manipulating Nucleic Acids

In alternative aspects, because the chimeric polypeptides used to practice this invention can be recombinantly generated, and can be used in recombinant form, the invention provides nucleic acids, which themselves can be recombinant, to make them. In alternative embodiments, nucleic acids of the invention are made, isolated and/or manipulated by, e.g., cloning and expression of cDNA libraries, amplification of message or genomic DNA by PCR, and the like.

The nucleic acids used to practice this invention, whether RNA, antisense nucleic acid, cDNA, genomic DNA, vectors, plasmids, viruses or hybrids thereof, can be isolated from a variety of sources, genetically engineered, amplified, and/or expressed/generated recombinantly. Recombinant polypeptides (e.g., the chimeric proteins used to practice this invention) generated from these nucleic acids can be individually isolated or cloned and tested for a desired activity. Any recombinant expression system can be used, including e.g. bacterial, fungal, mammalian, yeast, insect or plant cell expression systems.

Alternatively, nucleic acids used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Patent No. 4,458,066.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N. Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

The invention provides and uses chimeric proteins (also called “fusion proteins”) and nucleic acids encoding them. Any polypeptide used to practice this invention (e.g., chimeric protein) can be fused to a heterologous peptide or polypeptide, such as a peptide for targeting the polypeptide to a desired cell type, e.g., any cell involved in erythropoiesis. In alternative embodiments, a heterologous peptide or polypeptide joined or fused to a protein used to practice this invention can be an N-terminal identification peptide which imparts a desired characteristic, such as fluorescent detection, increased stability and/or simplified purification. Peptides and polypeptides used to practice this invention can also be synthesized and expressed as fusion proteins with one or more additional domains linked thereto for, e.g., producing a more immunogenic peptide, to more readily isolate a recombinantly synthesized peptide, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-414). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll (1993) DNA Cell. Biol., 12:441-53.

Nucleic acids used to practice this invention can be or comprise an oligonucleotide, nucleotide, polynucleotide, or a fragment of any of these, or DNA or RNA of genomic or synthetic origin, which may be single-stranded or double-stranded and may represent a sense or antisense strand, to peptide nucleic acid (PNA), or to any DNA-like or RNA-like material, natural or synthetic in origin. Compounds used to practice this invention include DNA or RNA (e.g., mRNA, rRNA, tRNA, iRNA) of genomic or synthetic origin which may be single-stranded or double-stranded; and can be a sense or antisense strand, or a peptide nucleic acid (PNA), or any DNA-like or RNA-like material, natural or synthetic in origin, including, e.g., iRNA, ribonucleoproteins (e.g., e.g., double stranded iRNAs, e.g., iRNPs). Compounds use to practice this invention include nucleic acids, i.e., oligonucleotides, containing known analogues of natural nucleotides. Compounds use to practice this invention include nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156. Compounds use to practice this invention include “oligonucleotides” including a single stranded polydeoxynucleotide or two complementary polydeoxynucleotide strands that may be chemically synthesized. Compounds use to practice this invention include synthetic oligonucleotides having no 5′ phosphate, and thus will not ligate to another oligonucleotide without adding a phosphate with an ATP in the presence of a kinase. A synthetic oligonucleotide can ligate to a fragment that has not been dephosphorylated.

In alternative aspects, compounds used to practice this invention include genes or any segment of DNA involved in producing a polypeptide chain (e.g., a chimeric protein of this invention); it can include regions preceding and following the coding region (leader and trailer) as well as, where applicable, intervening sequences (introns) between individual coding segments (exons). “Operably linked” can refer to a functional relationship between two or more nucleic acid (e.g., DNA) segments, e.g., an EPO specific protein and the EPO coding sequence, where a chimeric protein of the invention activates or upregulates the promoter to activate or upregulate transcription of the EPO coding sequence transcript; e.g., by binding to an EPO gene promoter. EPO gene promoters are well known in the art, see e.g., Tsuchiya (1997) J. Biochem. 121(2):193-196; Tarumoto (2000) Blood 96(5):1716-17122. EPO gene promoters have been cloned and characterized, e.g., in U.S. Patent App. Pub. No. 20080312179. Erythropoietin stimulating protein genes and promoters are also described e.g., in U.S. Patent App. Pub. Nos. 20060264377 and 20030166566.

In alternative aspects, it can refer to the functional relationship of transcriptional regulatory sequence to a transcribed sequence. For example, a promoter can be operably linked to a coding sequence, such as a nucleic acid used to practice this invention, if it stimulates or modulates the transcription of the coding sequence in an appropriate host cell or other expression system. In alternative aspects, promoter transcriptional regulatory sequences can be operably linked to a transcribed sequence where they can be physically contiguous to the transcribed sequence, i.e., they can be cis-acting. In alternative aspects, transcriptional regulatory sequences, such as enhancers, need not be physically contiguous or located in close proximity to the coding sequences whose transcription they enhance.

In alternative aspects, the invention comprises use of “expression cassettes” comprising a nucleotide sequence used to practice this invention, which can be capable of affecting expression of the nucleic acid, e.g., a structural gene or a transcript (e.g., encoding a chimeric polypeptide of this invention) in a host cell compatible with such sequences. Expression cassettes can include at least a promoter operably linked with the polypeptide coding sequence or inhibitory sequence; and, in one aspect, with other sequences, e.g., transcription termination signals. Additional factors necessary or helpful in effecting expression may also be used, e.g., enhancers.

In alternative aspects, expression cassettes used to practice this invention also include plasmids, expression vectors, recombinant viruses, any form of recombinant “naked DNA” vector, and the like. In alternative aspects, a “vector” used to practice this invention can comprise a nucleic acid (e.g., encoding a chimeric protein of this invention) that can infect, transfect, transiently or permanently transduce a cell. In alternative aspects, a vector used to practice this invention (e.g., comprising coding sequence for a chimeric protein of this invention) can be a naked nucleic acid, or a nucleic acid complexed with protein or lipid. In alternative aspects, vectors used to practice this invention can comprise viral or bacterial nucleic acids and/or proteins, and/or membranes (e.g., a cell membrane, a viral lipid envelope, etc.). In alternative aspects, vectors used to practice this invention can include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and can include both the expression and non-expression plasmids. In alternative aspects, the vector used to practice this invention can be stably replicated by the cells during mitosis as an autonomous structure, or can be incorporated within the host's genome.

In alternative aspects, “promoters” used to practice this invention include all sequences capable of driving transcription of a coding sequence in a cell, e.g., a mammalian cell such as a human cell. Thus, promoters used in the constructs of the invention include cis-acting transcriptional control elements and regulatory sequences that are involved in regulating or modulating the timing and/or rate of transcription of a sequence encoding a chimeric protein of this invention. For example, a promoter used to practice this invention can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription.

“Constitutive” promoters used to practice this invention can be those that drive expression continuously under most environmental conditions and states of development or cell differentiation. “Inducible” or “regulatable” promoters used to practice this invention can direct expression of the nucleic acid of the invention under the influence of environmental conditions, specific physiologic or pathologic conditions and/or developmental conditions.

### Kits and Instructions

The invention provides kits comprising compositions and methods of the invention, including instructions for use thereof. As such, kits, cells, vectors and the like can also be provided.

For example, in alternative embodiments, the invention provides kits comprising compositions comprising e.g., a set of (e.g., the plurality of) chimeric proteins of the invention; or a liquid or aqueous formulation of the invention; or a vesicle, liposome, nanoparticle or nanolipid particle of the invention. In one aspect, the kit further comprising instructions for practicing any composition or method of the invention, e.g., in vitro or ex vivo methods for directing in vitro, ex vivo or ex vivo synthesis of EPO in a mammalian cell.

### Formulations

In alternative embodiments, the invention provides compositions for use in in vitro, ex vivo or in vivo methods (including methods of the invention) for generating EPO message (transcripts) and/or proteins; including re-programming a mammalian cell to generate new or more EPO transcript or protein. In alternative embodiments, these compositions comprise a plurality of (a set of) proteins and/or nucleic acids formulated for these purposes, e.g., one or a plurality of chimeric proteins of the invention, and/or nucleic acids (e.g., vectors) that encode them, formulated in a buffer, in a saline solution, in a powder, an emulsion, in a vesicle, in a liposome, in a nanoparticle, in a nanolipoparticle and the like.

In alternative embodiments, the compositions can be formulated in any way and can be applied in a variety of concentrations and forms depending on the desired in vitro, ex vivo or in vivo conditions, a desired in vitro, ex vivo or in vivo method of administration and the like. Details on techniques for in vitro, ex vivo or in vivo formulations and administrations are well described in the scientific and patent literature.

Alternative formulations and/or carriers of the chimeric proteins or nucleic acids used to practice this invention are well known in the art. Formulations and/or carriers used to practice this invention can be in forms such as tablets, pills, powders, capsules, liquids, gels, syrups, slurries, suspensions, etc., suitable for in vitro or ex vivo applications.

In alternative embodiments chimeric proteins or nucleic acids used to practice this invention are in admixture with an aqueous and/or buffer solution or as an aqueous and/or buffered suspension, e.g., including a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate. Formulations can be adjusted for osmolarity, e.g., by use of an appropriate buffer.

For example, chimeric proteins of the invention can be formulated in sustained-release pharmaceutical formulations comprising carboxymethyl ether cellulose polymer, as described e.g., in U.S. Pat. No. 7,282,480. In one aspect, the sodium carboxymethyl ether cellulose is in a concentration range of about 0.5% to about 7% total formula weight. This pharmaceutical formulation can be administered e.g., thrice per two weeks, once per week, once per two weeks, once per three weeks, once per month, once per five weeks, or once per six weeks; e.g., at about 1 μg/ml to about 2000 μg/ml protein per formulation.

In alternative embodiments, oil-based formulations are used for in vitro, ex vivo or in vivo application of the compositions of the invention. Oil-based suspensions can be formulated by suspending the set of chimeric DRP proteins of the invention in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of hydrophobic compounds; see also U.S. Pat. No. 5,858,401. These formulations can be preserved by the addition of an antioxidant such as ascorbic acid. The formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. Formulations can also contain a buffer, preservative or a coloring agent.

In practicing this invention, the compounds (e.g., formulations) of the invention can comprise a solution of chimeric proteins or nucleic acids used to practice this invention dissolved in a pharmaceutically acceptable carrier, e.g., acceptable vehicles and solvents that can be employed include water and Ringer's solution, an isotonic sodium chloride. In addition, sterile fixed oils can be employed as a solvent or suspending medium. For this purpose any fixed oil can be employed including synthetic mono- or diglycerides, or fatty acids such as oleic acid. In one embodiment, solutions and formulations used to practice the invention are sterile and can be manufactured to be generally free of undesirable matter. In one embodiment, these solutions and formulations are sterilized by conventional, well known sterilization techniques.

The solutions and formulations used to practice the invention can comprise auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent (e.g., chimeric proteins) in these formulations can vary widely, and can be selected primarily based on fluid volumes, viscosities and the like, in accordance with the particular mode of in vitro, ex vivo or in vivo administration selected and the desired results, e.g., for de-differentiating or re-programming a mammalian cell.

The solutions and formulations used to practice the invention can be lyophilized; for example, the invention provides a stable lyophilized formulation comprising a chimeric protein or nucleic acid of the invention. In one aspect, this formulation is made by lyophilizing a solution comprising a chimeric protein or nucleic acid of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. patent app. no. 20040028670.

The compositions and formulations of the invention can be delivered by the use of liposomes (see also discussion, below). By using liposomes, particularly where the liposome surface carries ligands specific for target cells, or are otherwise preferentially directed to a specific tissue or organ type, one can focus the delivery of the active agent into a target cells in an in vitro or ex vivo application.

### Nanoparticles, Nanolipoparticles and Liposomes

The invention also provides nanoparticles, nanolipoparticles, vesicles and liposomal membranes comprising compounds used to practice the methods and compositions, e.g., a chimeric protein or nucleic acid of the invention, e.g., to deliver compositions of the invention to mammalian cells in vitro, ex vivo or in vivo. In alternative embodiments, these compositions are designed to target specific molecules, including biologic molecules, such as polypeptides, including cell surface polypeptides, e.g., for targeting a desired cell type, e.g., a mammalian cell targeted for de-differentiation or re-programming.

The invention provides multilayered liposomes comprising compounds used to practice this invention, e.g., as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition of this invention (e.g., a chimeric protein or nucleic acid of the invention).

Liposomes can be made using any method, e.g., as described in Park, et al., U.S. Pat. Pub. No. 20070042031, including method of producing a liposome by encapsulating an active agent (e.g., chimeric protein or nucleic acid of the invention), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice this invention comprise a substituted ammonium and/or polyanions, e.g., for targeting delivery of a compound (e.g., chimeric protein or nucleic acid) used to practice this invention to a desired cell type, as described e.g., in U.S. Pat. Pub. No. 20070110798.

The invention also provides nanoparticles comprising compounds (e.g., chimeric protein or nucleic acid) used to practice this invention in the form of active agent-containing nanoparticles (e.g., a secondary nanoparticle), as described, e.g., in U.S. Pat. Pub. No. 20070077286. In one embodiment, the invention provides nanoparticles comprising a fat-soluble active agent of this invention or a fat-solubilized water-soluble active agent to act with a bivalent or trivalent metal salt.

In one embodiment, solid lipid suspensions can be used to formulate and to deliver compositions of the invention to mammalian cells in vitro, ex vivo or in vivo, as described, e.g., in U.S. Pat. Pub. No. 20050136121.

### Polypeptide In Vitro, Ex Vivo or In Vivo Delivery Vehicles

In alternative embodiments, any delivery vehicle can be used to practice the methods or compositions of this invention, e.g., to deliver chimeric proteins of the invention to a host cell, e.g., a mammalian cell, in vitro, ex vivo or in vivo. For example, delivery vehicles comprising polycations, cationic polymers and/or cationic peptides, such as polyethyleneimine derivatives, can be used e.g. as described, e.g., in U.S. Pat. Pub. No. 20060083737.

In one embodiment, a dried polypeptide-surfactant complex is used to formulate a composition of the invention, wherein a surfactant is associated with a chimeric proteins of the invention via a noncovalent bond e.g. as described, e.g., in U.S. Pat. Pub. No. 20040151766.

In one embodiment, a covalent conjugate between a poly(alkylene oxide) and a glycosylated or non-glycosylated chimeric protein of the invention is used, where a poly(alkylene oxide) can be conjugated to a chimeric protein via a glycosyl linking group, and a glycosyl linking group can be interposed between a chimeric protein and a poly(alkylene oxide). A covalent conjugate can be formed by contacting a chimeric protein with a glycosyltransferase and a modified sugar donor; the glycosyltransferase transfers the modified sugar moiety to the chimeric protein to form a covalent conjugate; the modified sugar moiety can be a poly(alkylene oxide). See e.g., U.S. Pat. No. 7,416,858.

In one embodiment, a chimeric protein used to practice this invention can be applied to cells as polymeric hydrogels or water-soluble copolymers, e.g., as described in U.S. Pat. No. 7,413,739; for example, a chimeric protein can be polymerized through a reaction between a strong nucleophile and a conjugated unsaturated bond or a conjugated unsaturated group, by nucleophilic addition, wherein each precursor component comprises at least two strong nucleophiles or at least two conjugated unsaturated bonds or conjugated unsaturated groups. In one embodiment, a chimeric protein used to practice this invention can be applied to cells using vehicles with cell membrane-permeant peptide conjugates, e.g., as described in U.S. Pat. Nos. 7,306,783; 6,589,503. In one aspect, the chimeric protein itself is conjugated to a cell membrane-permeant peptide. In one embodiment, a chimeric protein and/or the delivery vehicle are conjugated to a transport-mediating peptide, e.g., as described in U.S. Pat. No. 5,846,743, describing transport-mediating peptides that are highly basic and bind to poly-phosphoinositides.

In one embodiment, electro-permeabilization is used as a primary or adjunctive means to deliver a chimeric protein of the invention to a cell, e.g., using any electroporation system as described e.g. in U.S. Pat. Nos. 7,109,034; 6,261,815; 5,874,268.

### Pharmaceutical Compositions

The invention provides compositions for ameliorating anemia and/or stimulating EPO synthesis and EPO synthesis, including pharmaceutical compositions, e.g., in the manufacture of medicaments for ameliorating anemia and/or stimulating erythropoiesis and EPO synthesis.

In alternative embodiments, the compositions (e.g., comprising chimeric proteins and/or nucleic acids encoding chimeric proteins) of the invention are formulated with a pharmaceutically acceptable carrier. In alternative embodiments, the pharmaceutical compositions of the invention can be administered subcutaneously, parenterally, topically, orally or by local administration, such as by aerosol or transdermally. The pharmaceutical compositions can be formulated in any way and can be administered in a variety of unit dosage forms depending upon the condition (e.g., anemia) or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”).

Therapeutic agents of the invention can be administered alone or as a component of a pharmaceutical formulation (composition). The compounds may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions (e.g., chimeric proteins) of the invention include those suitable for subcutaneous, spray, nebulized, oral/nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single or multiple dosage form will vary depending upon the host being treated, the particular mode of administration. The amount of active ingredient which can be combined with a carrier material to produce a single or multiple dosage form will generally be that amount of the compound which produces a therapeutic effect.

Pharmaceutical formulations of this invention can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for liquid or oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, e.g., push-fit capsules made of gelatin, as well as soft, sealed capsules made of gelatin and a coating such as glycerol or sorbitol. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., a chimeric polypeptide or peptidomimetic of the invention) in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil-based pharmaceuticals are particularly useful for administration of the compositions (e.g., chimeric proteins) of the invention. Oil-based suspensions can be formulated by suspending an active agent (e.g., chimeric protein of the invention) in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In practicing this invention, the pharmaceutical compounds can also be administered by in intranasal, intraocular and intravaginal routes including suppositories, insufflation, powders and aerosol formulations (for examples of steroid inhalants, see Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In practicing this invention, the pharmaceutical compounds can be delivered by transdermally, by a topical route, and/or can be formulated as applicator sticks, solutions, suspensions, emulsions, gels, creams, ointments, pastes, jellies, paints, powders, and aerosols. In alternative embodiments, topical formulations of the invention comprising e.g., chimeric proteins of the invention and/or nucleic acids encoding them, comprise an ointment, a cream, a powder, an emulsion, a gel, a glycerogelatin (a preparation made from glycerin and glycerinated gelatin), a paste, a plaster, a sprayable composition or a lotion.

In alternative embodiments, pharmaceutical compositions of the invention are formulated as cream formulation, e.g., containing no or up to 12% urea, e.g., comprising aqua, caprylic and/or capric triglyceride, pentylene glycol, hydrogenated lecithin, butyrospermum parkii, glycerin, squalane, ceramide 3 and the like. In alternative embodiments, pharmaceutical compositions of the invention are formulated as water based or non-water based suspensions or pastes. In alternative embodiments, pharmaceutical compositions of the invention are formulated moist or dry, and can be combined with adjuvants and/or form ointments, creams, powders, emulsions, lotions, gels, pastes, plasters, glycerogelatiness or sprays. In alternative embodiments, ointments in the form of semisolid preparation are used for external applications, e.g., to skin or mucous membranes. In alternative embodiments, ointments can be prepared by incorporation or by fusion; or the ointments can comprise hydrocarbon bases, e. g. petrolatum USP, white petrolatum USP, yellow ointment USP, or white ointment USP, absorption bases, e. g. hydrophilic petrolatum USP, or lanolin USP, water-removable bases, e. g. hydrophilic ointment USP, water-soluble bases, e. g. a polyethylene glycol ointment.

In alternative embodiments, creams are formulated as semisolid preparations, and compositions of the invention can be dissolved or dispensed in either an oil-in-water emulsion or in another type of water-washable base. Creams also can be used rectally and/or vaginally. Gels are semisolid systems consisting of dispersions of large molecules in an aqueous liquid vehicle rendered jelly-like through the addition of a gelling agent. Examples of gelling agents are carbomer 934, cellulose derivatives as carboxymethylcellulose or hydroxypropylmethyl-cellulose, and natural gums as tragacanth. Carbomers are high molecular weight water-soluble polymers of acrylic acid cross-linked with allyl ethers of sucrose and/or pentaerythritol.

In alternative embodiments, pastes are formulated as semisolid preparations for application to the skin or mucous membranes. Pastes may comprise zinc oxide, starch and/or white petrolatum. In alternative embodiments, plasters are formulated as solid or semi-solid adhesive masses spread upon a backing material of paper, fabric, moleskin or plastic. The adhesive material can be a rubber base or synthetic resin. Plasters can be applied to skin to provide prolonged contact at the site. In alternative embodiments, glycerogelatins comprise gelatin, glycerol and water.

In alternative embodiments, sprays are formulated as aqueous or oleaginous solutions in the form of coarse droplets or as finely divided solids to be applied topically, most usually to the nasal-pharyngeal tract or to the skin. Any mechanical device for spraying or nebulizing can be used in practicing the invention.

In practicing this invention, the pharmaceutical compounds also can be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In practicing this invention, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs.

For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

Pharmaceutical compositions of this invention can be formulated in a liquid form, and can be injected intravenously or subcutaneously. In one embodiment, a composition of this invention, e.g., comprising a chimeric polypeptide of this invention, is administered with EPO.

Pharmaceutical compositions of this invention can be formulated as stable liquid preparations, e.g., as described in U.S. Pat. App. Pub. No. 20080039371, where the formulation would comprise a chimeric polypeptide of this invention and amino acids, e.g., leucine, isoleucine, threonine, glutamic acid, aspartic acid and/or phenyl alanine or any combination thereof; where in one embodiment the formulation is free of preservatives, urea and HAS. In alternative embodiments the formulation has an osmolality about 200 to 400 mosmol/kg, or 250 to 300 mosmol/kg, or 260 to 290 mosmol/kg. Pharmaceutical compositions of this invention can be formulated as described e.g. in U.S. Pat. App. Pub. No. 20070293419, using e.g., a non-ionic surfactant, polyhydric alcohol, neutral amino acid and sugar alcohol as stabilizers; isotonic reagent; and/or a buffering reagent. Pharmaceutical compositions of this invention can be formulated as described e.g. in U.S. Pat. App. Pub. No. 20070128231, using e.g., a pH buffering agent a sodium phosphate buffer; as a stabilizer tris-(hydroxymethyl)-aminomethane; and/or a pharmaceutically effective quantity of a chimeric polypeptide or nucleic acid of this invention.

The compositions and formulations of the invention can be delivered by the use of liposomes (see also discussion, below). By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587.

The formulations of the invention can be administered for prophylactic and/or therapeutic treatments. In therapeutic applications, compositions are administered to a subject already suffering from a condition, infection or disease in an amount sufficient to cure, alleviate or partially arrest the clinical manifestations of the condition, infection or disease and its complications (a “therapeutically effective amount”). For example, in alternative embodiments, pharmaceutical compositions of the invention are administered in an amount sufficient to treat, prevent and/or ameliorate anemia or less than normal levels of erythropoiesis. The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate. For example, in one embodiment a therapeutically effective amount of a chimeric polypeptide of this invention is a dosage of between about 0.025 to 0.5 milligram per 1 kilogram of body weight of the patient; or, a therapeutically effective amount is a dosage of between about 0.025 to 0.2 milligram, or 0.05 to 0.1 milligram, or 0.075 to 0.5 milligram, or 0.2 to 0.4 milligram, of the compound per 1 kilogram of body weight of the patient.

Single or multiple administrations of formulations can be given depending on the dosage and frequency as required and tolerated by the patient. The formulations should provide a sufficient quantity of active agent to effectively treat, prevent or ameliorate a conditions, diseases or symptoms as described herein. For example, an exemplary pharmaceutical formulation for oral administration can be in a daily amount of between about 0.1 to 0.5 to about 20, 50, 100 or 1000 or more μg per kilogram of body weight per day of chimeric protein. In an alternative embodiment, dosages are from about 1 mg to about 4 mg per kg of body weight per patient per day of chimeric protein are used. Lower dosages can be used, in contrast to administration by inhalation (e.g., bys spray or nebulizer), orally, into the blood stream, into a body cavity or into a lumen of an organ. Substantially higher dosages can be used in subcutaneous, topical or oral administration or administering by nebulizers, powders, spray or inhalation. Actual methods for preparing parenterally or non-parenterally administrable formulations will be known or apparent to those skilled in the art and are described in more detail in such publications as Remington's, supra.

The methods of the invention can further comprise co-administration with other drugs or pharmaceuticals, e.g., compositions for treating anemia and related symptoms or conditions. For example, the methods and/or compositions and formulations of the invention can be co-formulated with and/or co-administered with antibiotics (e.g., antibacterial or bacteriostatic peptides or proteins), including those effective against gram negative bacteria, fluids, cytokines, immunoregulatory agents, anti-inflammatory agents, complement activating agents, such as peptides or proteins comprising collagen-like domains or fibrinogen-like domains (e.g., a ficolin), carbohydrate-binding domains, and the like and combinations thereof.

### Nanoparticles and Liposomes

The invention also provides nanoparticles and liposomal membranes comprising compounds of this invention which target specific molecules, including biologic molecules, such as polypeptide, including cell surface polypeptides. Thus, in alternative embodiments, the invention provides nanoparticles and liposomal membranes targeting EPO-producing cells, or any cell comprising an EPO protein coding sequence (which as described above can be a heterologous EPO protein coding sequence.

In alternative embodiments, the invention provides nanoparticles and liposomal membranes comprising (in addition to comprising compounds of this invention) molecules, e.g., peptides or antibodies, that selectively target EPO-producing cells or any cell comprising a heterologous EPO protein coding sequence. In alternative embodiments, the invention provides nanoparticles and liposomal membranes using receptors to target EPO-producing cells or any cell comprising a heterologous EPO protein coding sequence.

Thus, in one aspect, the compositions of the invention are specifically targeted for inhibiting, ameliorating and/or preventing anemia, and/or increasing levels of EPO produced by a cell and/or individual.

The invention also provides nanocells to allow the sequential delivery of two different therapeutic agents with different modes of action or different pharmacokinetics, at least one of which comprises a composition of this invention. A nanocell is formed by encapsulating a nanocore with a first agent inside a lipid vesicle containing a second agent; see, e.g., Sengupta, et al., U.S. Pat. Pub. No. 20050266067. The agent in the outer lipid compartment is released first and may exert its effect before the agent in the nanocore is released. The nanocell delivery system may be formulated in any pharmaceutical composition for delivery to patients suffering from a diseases or condition related to anemia. In treating cancer, a traditional antineoplastic agent is contained in the outer lipid vesicle of the nanocell, and an agent of this invention is loaded into the nanocore.

The invention also provides multilayered liposomes comprising compounds of this invention, e.g., for transdermal absorption, e.g., as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition of this invention.

A multilayered liposome of the invention may further include an antiseptic, an antioxidant, a stabilizer, a thickener, and the like to improve stability. Synthetic and natural antiseptics can be used, e.g., in an amount of 0.01% to 20%. Antioxidants can be used, e.g., BHT, erysorbate, tocopherol, astaxanthin, vegetable flavonoid, and derivatives thereof, or a plant-derived antioxidizing substance. A stabilizer can be used to stabilize liposome structure, e.g., polyols and sugars. Exemplary polyols include butylene glycol, polyethylene glycol, propylene glycol, dipropylene glycol and ethyl carbitol; examples of sugars are trehalose, sucrose, mannitol, sorbitol and chitosan, or monosaccharides or oligosaccharides, or a high molecular weight starch. A thickener can be used for improving the dispersion stability of constructed liposomes in water, e.g., a natural thickener or an acrylamide, or a synthetic polymeric thickener. Exemplary thickeners include natural polymers, such as acacia gum, xanthan gum, gellan gum, locust bean gum and starch, cellulose derivatives, such as hydroxy ethylcellulose, hydroxypropyl cellulose and carboxymethyl cellulose, synthetic polymers, such as polyacrylic acid, poly-acrylamide or polyvinylpyrollidone and polyvinylalcohol, and copolymers thereof or cross-linked materials.

Liposomes can be made using any method, e.g., as described in Park, et al., U.S. Pat. Pub. No. 20070042031, including method of producing a liposome by encapsulating a therapeutic product comprising providing an aqueous solution in a first reservoir; providing an organic lipid solution in a second reservoir, wherein one of the aqueous solution and the organic lipid solution includes a therapeutic product; mixing the aqueous solution with said organic lipid solution in a first mixing region to produce a liposome solution, wherein the organic lipid solution mixes with said aqueous solution so as to substantially instantaneously produce a liposome encapsulating the therapeutic product; and immediately thereafter mixing the liposome solution with a buffer solution to produce a diluted liposome solution. The invention also provides nanoparticles comprising compounds of this invention to deliver a composition of the invention as a drug-containing nanoparticles (e.g., a secondary nanoparticle), as described, e.g., in U.S. Pat. Pub. No. 20070077286. In one embodiment, the invention provides nanoparticles comprising a fat-soluble drug of this invention or a fat-solubilized water-soluble drug to act with a bivalent or trivalent metal salt.

### Products of Manufacture, Implants and Artificial Organs

The invention also provides products of manufacture comprising cells of the invention (e.g., cells comprising coding sequence for chimeric polypeptides of this invention), and use of cells made by methods of this invention, including for example implants and artificial organs, bioreactor systems, cell culture systems, plates, dishes, tubes, bottles and flasks comprising cells of this invention. Any implant, artificial organ, bioreactor systems, cell culture system, cell culture plate, dish (e.g., petri dish), cell culture tube and/or cell culture flask (e.g., a roller bottle) can be used to practice this invention.

In alternative embodiments the invention provides a bioreactor, implant, stent, artificial organ or similar device comprising a cell of the invention, or cells made by a method of this invention; for example, including implants as described in U.S. Pat. Nos. 7,388,042; 7,381,418; 7,379,765; 7,361,332; 7,351,423; 6,886,568; 5,270,192; and U.S. Pat. App. Pub. Nos. 20040127987; 20080119909 (describing auricular implants); 20080118549 (describing ocular implants); 20080020015 (describing a bioactive wound dressing); 20070254005 (describing heart valve bio-prostheses, vascular grafts, meniscus implants); 20070059335; 20060128015 (describing liver implants).

### Implanting Cells In Vivo

In alternative embodiments, the methods of the invention also comprise implanting or engrafting cells of the invention (e.g., cells comprising coding sequence for chimeric polypeptides of this invention) in a vessel, tissue or organ; and in one aspect, comprise implanting or engrafting these cells in a vessel, tissue or organ ex vivo or in vivo, or implanting or engrafting these cells in an individual in need thereof.

Cells can be removed from an individual, treated using the compositions and/or methods of this invention, and reinserted (e.g., injected or engrafted) into a tissue, organ or into the individual, using any known technique or protocol. For example, de-differentiated re-programmed cells, or re-programmed differentiated cells, can be re-implanted (e.g., injected or engrafted) using microspheres e.g., as described in U.S. Pat. No. 7,442,389; e.g., in one aspect, the cell carrier comprises a bulking agent comprising a plurality of round and smooth polymethylmethacrylate microparticles preloaded within a mixing and delivery system and an autologous carrier comprising these cells. In another embodiment, the cells are readministered to a tissue, an organ and/or an individual in need thereof in a biocompatible crosslinked matrix, as described e.g., in U.S. Pat. App. Pub. No. 20050027070.

In another embodiment, the cells of the invention (e.g., cells made by practicing the methods of this invention) are readministered (e.g., injected or engrafted) to a tissue, an organ and/or an individual in need thereof within, or protected by, a biocompatible, nonimmunogenic coating, e.g., as on the surface of a synthetic implant, e.g., as described in U.S. Pat. No. 6,969,400, describing e.g., a protocol where a chimeric protein of this invention can be conjugated to a polyethylene glycol that has been modified to contain multiple nucleophilic groups, such as primary amino or thiol group.

In one embodiment, the cells of the invention (e.g., cells made by practicing the methods of this invention; cells comprising coding sequence for chimeric polypeptides of this invention) are readministered (e.g., injected or engrafted) to a tissue, an organ and/or an individual in need thereof using grafting methods as described e.g. by U.S. Pat. Nos. 7,442,390; 5,733,542.

### Diagnosing and Treating Anemia

In alternative embodiments, the invention provides methods for ameliorating or preventing an anemia, and/or stimulating erythropoiesis and/or erythropoietin (EPO) synthesis, in an individual. In practicing the invention any diagnostic method for determining when anemia is present, or the severity of the anemia, or whether the anemia is responding to treatment (e.g., responding to administration of a chimeric protein of the invention, or administration of a nucleic acid encoding a chimeric protein of the invention), or whether levels of EPO are normal or responding to treatment, can be used.

For example, because alternative embodiments of the invention encompass ameliorating or preventing anemia caused by a genetic disorder, an infection, a dietary disorder or deficiency, a pollutant, a pesticide, herbicide or insecticide, a poison, a venom, a toxin, a biological agent, a drug, a cancer or a cancer therapeutic or cancer therapy, the presence of any of these conditions can be used as a basis for diagnosing or predicting the possible onset of anemia, and any diagnostic technique related to any of these conditions, treatments, infections or exposures can be used.

For example, because alternative embodiments of the invention encompass ameliorating or preventing anemia caused by a drug-induced anemia; caused by an infection; caused by an iron deficiency; caused by rhesus disease (hemolytic disease of newborn): caused by sickle-cell disease, thalassemia or Plummer-Vinson syndrome (PVS); a sideroblastic anemia-congenital or acquired; caused by Gaucher's disease; caused by a vitamin deficiency; caused by autoimmune hemolytic anemia (AIHA); caused by a cancer; or, caused by heavy metal poisoning or pyridoxine deficiency, any diagnostic test for any of these conditions, treatments, infections or exposures can be used.

For example, anemia can be diagnosed using complete blood counts, the number of red blood cells and/or the hemoglobin level. Automatic counters that measure the size of the red blood cells, e.g., by flow cytometry, can be used to distinguish between the causes of anemia; noting that in alternative embodiments the compositions and methods of the invention are used to ameliorate or prevent microcytic, normocytic or macrocytic forms of anemia. Examination of a stained blood smears, e.g., using a microscope, also can be used. In alternative embodiments, four parameters are used to diagnose or assess anemia: RBC count, hemoglobin concentration, mean corpuscular volume (MCV) and red blood cell distribution width (RDW); e.g., to access the individual's response to administration of a chimeric protein of the invention, or administration of a nucleic acid encoding a chimeric protein of the invention. Hematocrit, mean corpuscular hemoglobin (or “mean cell hemoglobin”, MCH), and mean corpuscular hemoglobin concentration (or MCHC) also can be calculated, and compared to values adjusted for age and sex. For example, for adult men a hemoglobin level less than 13.0 g/dl (grams per deciliter) is diagnostic of anemia, and for adult women, the diagnostic threshold is below 12.0 g/dl. A normal hematocrit value is 32 to 36 g/dl.

In alternative embodiments, other tests also can be used, e.g., the erythrocyte sedimentation rate (ESR); the reticulocyte production index (RPI, also called a corrected reticulocyte count); folic acid, serum iron, hepcidin, transferrin, vitamin B12 and/or creatinine levels; e.g., to access the individual's response to administration of a chimeric protein of the invention, or administration of a nucleic acid encoding a chimeric protein of the invention.

In alternative embodiments, methods of this invention can be practiced with other treatments for anemia, e.g., blood transfusions, hyperbaric medicine, administration of an EPO, e.g., a recombinant erythropoietin; for example, any form of erythropoietin can be used, including epoetin (e.g., EPOGEN™, PROCRIT™, EPREX™, NEORECORMON™) (Amgen, Thousand Oaks, Calif.), or darbepoetin, a synthetic form of EPO (e.g., ARANESP™) (Amgen, Thousand Oaks, Calif.). In alternative embodiments, methods of this invention can be practiced with other treatments for anemia such as administering a VEGF antagonist, as described e.g., in U.S. Pat. No. 7,351,411; administering a hyperglycosylated analog of EPO, as described e.g., in U.S. Pat. No. 7,304,150 or U.S. Pat. No. 7,262,166.

Treating Conditions Response to Increasing EPO Production

In alternative embodiments, the invention also provides methods for increasing endogenous erythropoietin (EPO) production in an individual in need thereof using compositions of this invention; e.g., including pharmaceutical or sterile formulations, the chimeric protein of the invention; liquids, gels, hydrogels, powders or aqueous formulations of the invention; vesicles, liposomes, nanoparticles or nanolipid particles (NLP) of the invention; isolated or cultured cells of the invention; nucleic acids of the invention; or vectors, plasmid, recombinant virus or expression vehicle of the invention.

Individuals that can benefit by practicing the compositions and methods of this invention, e.g., that can benefit (e.g., by treating, ameliorating and/or preventing) from an increase in endogenous erythropoietin (EPO) production, including humans or animals, include e.g., individuals with a peripheral nerve injury; a hemoglobin H disease; a testicular torsion-detorsion; post-infarct myocardial damage; or a neurologic disorder; or an increase in endogenous EPO levels would act as a neuroprotective, e.g., in low birth weight infants. In alternative embodiments, the neurologic disorder is ischemic stroke, intracerebral hemorrhage, subarachnoid hemorrhage, traumatic brain injury or Parkinson's disease.

Wounds and inflammations also can be treated or ameliorated by practicing the compositions and methods of this invention, e.g., including humans or animals. In alternative embodiments, wounds and inflammations that can benefit from an increase in endogenous erythropoietin (EPO) production by practicing the compositions and methods of this invention include conjunctivitis; surgical or accidental wounds; a bedsore; a burn; an inflammation of the skin, mucous membranes, airways or lungs; an eczema or a skin disorder accompanied by necrosis, by dermatitis, by psoriasis or by diabetes mellitus. The pharmaceutical formulation applied can be a topical application, e.g., in the form of an ointment, a cream, a powder, an emulsion, a gel, a glycerogelatin, a paste, a plaster, a sprayable composition or a lotion.

The invention will be further described with reference to the following examples; however, it is to be understood that the invention is not limited to such examples.

### EXAMPLES

### Example 1

### Compositions and Methods of the Invention are Effective in the Amelioration of Anemia

The data presented herein demonstrates that the compositions and methods of the invention are effective in the amelioration of anemia.

In one embodiment, each finger of a chimeric protein (Designed Regulatory Protein, or DRP) of the invention is connected by a flexible linker having the amino acid sequence TGEKP, this motif is highly conserved among C2H2 zinc fingers. Exactly the same scaffold was used for each finger except for DNA binding residues. Listed below is are exemplary chimeric polypeptides of this invention: the so-called “EPO1-DRP” (SEQ ID NO:1); and, the so-called “EPO2-DRP” (SEQ ID NO:2):

Amino acid sequence the exemplary DRP-1
of this invention

```
MGRRRRRRRRRGGGPKKKRKVGGGGSTGEKPYKCPECGKSFS [symbol] S [symbol] LQ [symbol] H
          R-CPP NLS Finger-1
QRTH-
TGEKPYKCPECGKSFS [symbol] S [symbol] LQ [symbol] HQRTHTGEKPYKCPECGKSFS [symbol] S [symbol] LQ
                          Finger-2 Finger-3 [symbol]
HQRTH-
TGEKPYKCPECGKSFS [symbol] S [symbol] LQ [symbol] HQRTHTGEKPYKCPECGKSFS [symbol] S [symbol] LQ
                          Finger-4 Finger-5 [symbol]
HQRTH-
TGEKPYKCPECGKSFS [symbol] S [symbol] LQ [symbol] HQRTHGGGGSPRPTDVSLGDELHLDGE
                          Finger-6 VP16
DVAMAH-
ADADLLFDLDMLGDGDSPGPGFTPHDSAPYGALDMADFEFEQMFTDALG
                          VP16
DYKDDDDK
FLAG-tag
```

Amino acid sequence of the exemplary DRP-2
of this invention

```
MGRRRRRRRRRGGGPKKKRKVGGGGSTGEKPYKCPECGKSFS [symbol] S [symbol] LQ [symbol] H
          R9-CPP NLS Finger-1
QRTH-
TGEKPYKCPECGKSFS [symbol] S [symbol] LQ [symbol] HQRTHTGEKPYKCPECGKSFS [symbol] S [symbol] LQ
                          Finger-2 Finger-3 [symbol]
HQRTH-
TGEKPYKCPECGKSFS [symbol] S [symbol] LQ [symbol] HQRTHTGEKPYKCPECGKSFS [symbol] S [symbol] LQ
                          Finger-4 Finger-5 [symbol]
HQRTH-
TGEKPYKCPECGKSFS [symbol] S [symbol] LQ [symbol] HQRTHGGGGSPRPTDVSLGDELHLDGE
                          Finger-6 VP16
DVAMAH-
ADALDDFDLDMLGDGDSPGPGFTPHDSAPYGALDMADFEFEQMFTDALG
                          VP16
DYKDDDDK
FLAG-tag
```

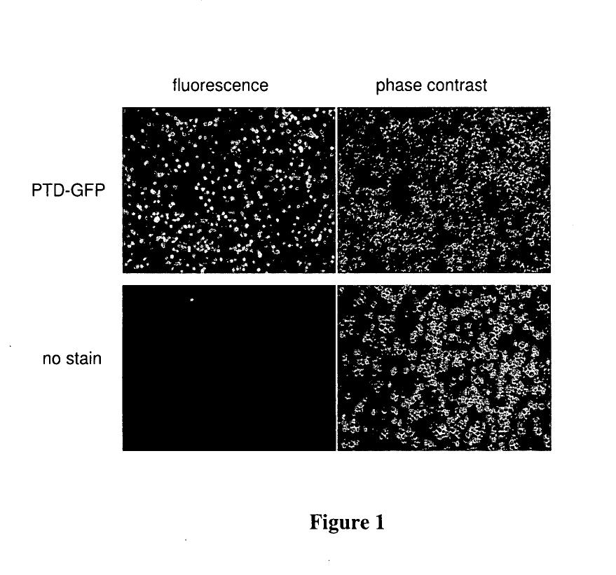

Normal human lymphocytes were used, and the cells contacted with chimeric proteins of this invention, the so-called “Designed Regulatory Proteins (DRPs)” of the invention, using a procedure that is the same as described in Tachikawa, K. et al. Proc. Natl. Acad. Sci. USA 101:15225-30, 2004. Purified EPO1-DRP (SEQ ID NO:1) or EPO2-DRP (SEQ ID NO:2) protein was incubated with the cells. FIG. 1 illustrates photomicrograph images that confirms that chimeric proteins (DRPs) of this invention can enter mammalian cells, using in this example human lymphocyte cells: left two panels illustrate a fluorescence image, and right two panels illustrate a phase contrast image; the upper two panels illustrate detection of green fluorescent protein expressed by a DRP of the invention.

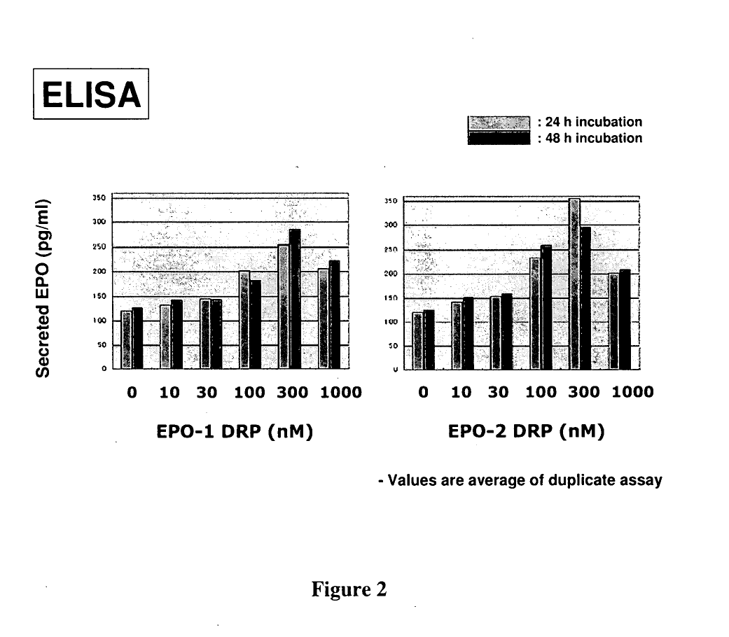

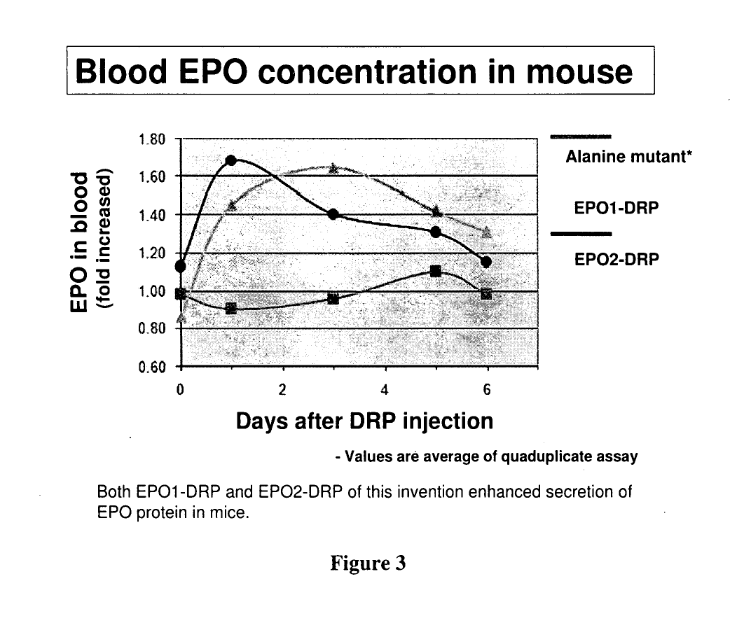

FIG. 2 illustrates data from an ELISA assay demonstrating that both EPO1-DRP (the DRP-1 described above, SEQ ID NO:1) and EPO2-DRP (the DRP-2 described above, SEQ ID NO:2) of this invention when inserted into mammalian cells can enhance secretion of EPO protein in those cells. For example, as graphically illustrated in FIG. 2, both EPO1-DRP and EPO2-DRP enhanced secretion of EPO protein in mouse fibroblast cells. FIG. 3 graphically illustrates data of blood EPO in these mice (i.e., animal administered EPO1-DRP or EPO2-DRP) further demonstrating both EPO1-DRP and EPO2-DRP of this invention when inserted into mammalian cells can enhance secretion of EPO protein in those cells. Both the exemplary EPO1-DRP and EPO2-DRP caused an enhancement of secretion of EPO in the mice. In FIG. 3, the “*” indicates exemplary alanine mutants: the figure illustrating amino acid residues necessary for sequence specific recognition (i.e. −1, +2, +3, and +6 in the each finger domain of the DRP) were replaced with alanine (ala).

### Example 2

### Designing Compositions of the Invention to Ameliorate Anemia

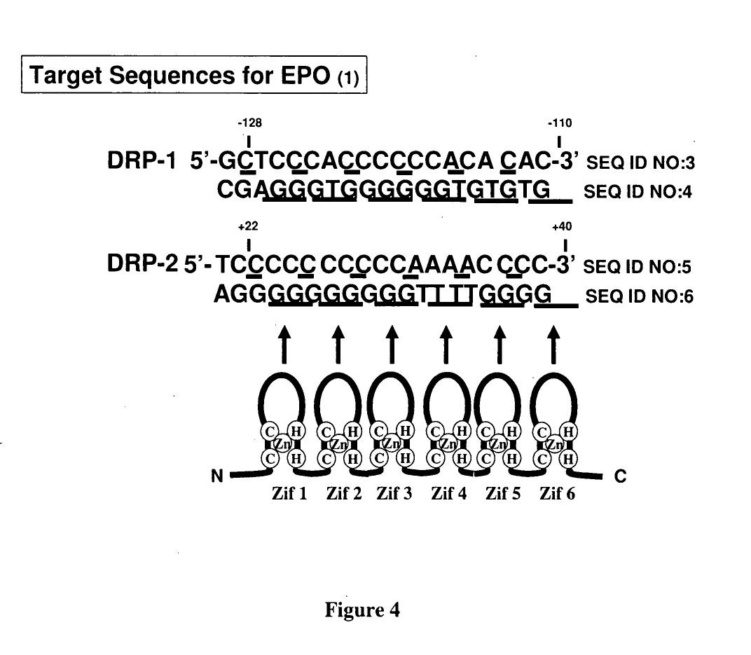

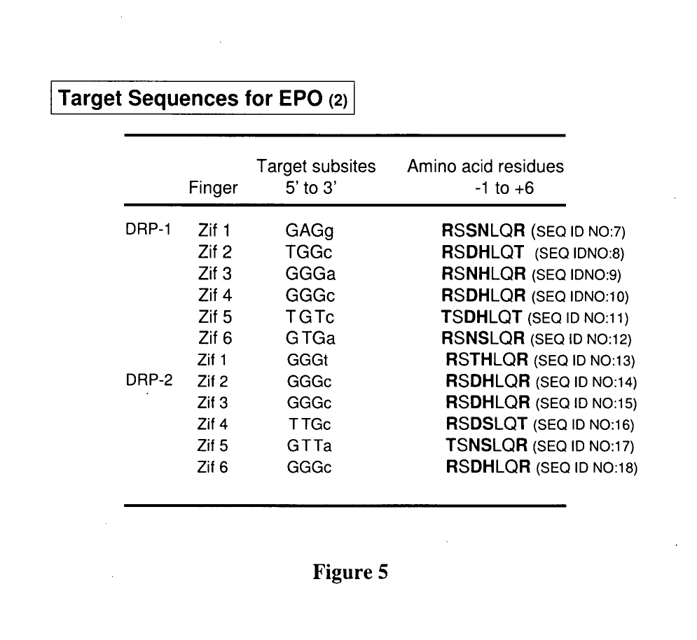

FIG. 4 illustrates exemplary zinc finger DNA target sequences for chimeric proteins (DRPs) of the invention, including but not limited to the illustrated DRP-1 and DRP-2 target site illustrated in this figure; “*” indicates the number on the zinc finger target sequence that represents the position relative to the transcription start site. FIG. 5 illustrates alternative exemplary zinc finger DNA target sequences for chimeric proteins (DRPs) of the invention, with the corresponding exemplary zinc finger DNA-binding motif; the figure illustrates exemplary zinc finger DNA target subsites 5′ to 3′ and amino acid residues −1 to +6 (minus one to plus six):

| | Target subsites | | Amino acid residues | |
|---|---|---|---|---|
| Finger | 5′ to 3′ | | −1 to +6 | |
| Zif 1 | GAGg | (SEQ ID NO: 19) | RSSNLQR | (SEQ ID NO: 7) |
| Zif 2 | TGGc | (SEQ ID NO: 20) | RSDHLQT | (SEQ ID NO: 8) |
| Zif 3 | GGGa | (SEQ ID NO: 21) | RSNHLQR | (SEQ ID NO: 9) |
| Zif 4 | GGGc | (SEQ ID NO: 22) | RSDHLQR | (SEQ ID NO: 10) |
| Zif 5 | T G Tc | (SEQ ID NO: 23) | TSDHLQT | (SEQ ID NO: 11) |
| Zif 6 | G TGa | (SEQ ID NO: 24) | RSNSLQR | (SEQ ID NO: 12) |
| Zif 1 | GGGt | (SEQ ID NO: 25) | RSTHLQR | (SEQ ID NO: 13) |
| Zif 2 | GGGc | (SEQ ID NO: 26) | RSDHLQR | (SEQ ID NO: 14) |
| Zif 3 | GGGc | (SEQ ID NO: 27) | RSDHLQR | (SEQ ID NO: 15) |
| Zif 4 | T TGc | (SEQ ID NO: 28) | RSDSLQT | (SEQ ID NO: 16) |
| Zif 5 | G T Ta | (SEQ ID NO: 29) | TSNSLQR | (SEQ ID NO: 17) |
| Zif 6 | GGGc | (SEQ ID NO: 30) | RSDHLQR | (SEQ ID NO: 18) |

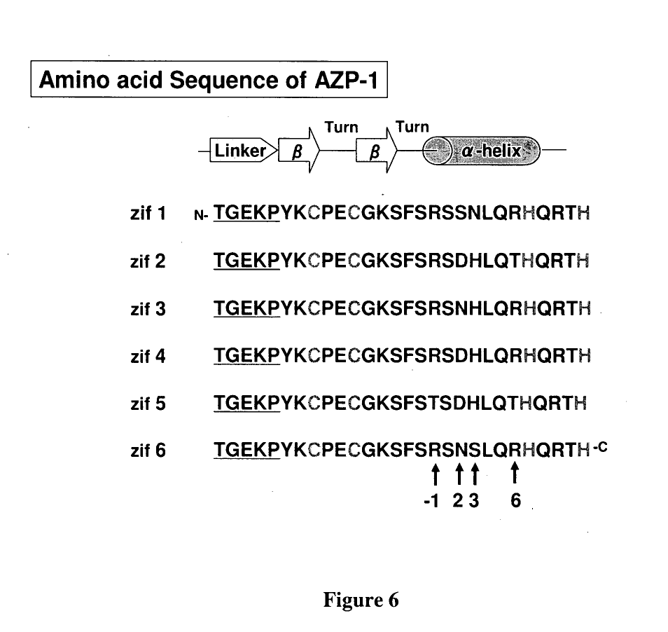

This invention can incorporate any zinc finger-interacting protein family member, or “zif”, including the zif motifs illustrated in FIG. 6, including the exemplary zif-1, zif-2, zif-3, zif-4, zif-5 and/or zif-6.

This invention can incorporate any protein that can specifically bind to a zinc finger target sequence, e.g., as described in U.S. Pat. App. Pub. Nos. 20030082561, 20030134350 and 20040091878 (also WO/2003/062455), describing a context-independent recognition code to design zinc finger domains; the code permits identification of an amino acid for positions −1, 2, 3 and 6 of the alpha-helical region of the zinc finger domain from four-base pair nucleotide target sequences.

This invention can incorporate any zinc finger binding protein or motif, e.g., as described in U.S. Pat. App. Pub. No. 20080269471; or U.S. Pat. App. Pub. No. 20080182332, describing zinc fingers comprising CCHC zinc coordinating residues; or U.S. Pat. App. Pub. No. 20060246588, describing non-canonical (e.g., non-C₂H₂) zinc finger proteins.

For example, embodiments of the invention include chimeric proteins comprising one or more or all of the following amino acid zinc finger DNA binding motifs (see also FIG. 6):

| | |
|---|---|
| TGEKPYKCPECGKSFSRSSNLQRHQRTH | (SEQ ID NO: 31) |
| TGEKPYKCPECGKSFSRSDHLQTHQRTH | (SEQ ID NO: 32) |
| TGEKPYKCPECGKSFSRSNHLQRHQRTH | (SEQ ID NO: 33) |
| TGEKPYKCPECGKSFSRSDHLQRHQRTH | (SEQ ID NO: 34) |
| TGEKPYKCPECGKSFSTSDHLQTHQRTH | (SEQ ID NO: 35) |
| TGEKPYKCPECGKSFSRSNSLQRHQRTH | (SEQ ID NO: 36) |

Chimeric proteins of this invention can be designed to specifically bind to, and modulate (e.g., activate or inhibit) the transcription of, any EPO gene, for example:

| LOCUS EPO-6\in\p 957 bp DNA 6 JUL. 2006 | |
|---|---|
| COMMENT CGexDoc “EPO-6 in pET21” | |
| ORIGDB\|GenBank | |
| BASE COUNT 262 a 236 c 267 g 192 t | |
| (SEQ ID NO: 37) | |
| 1 | taattttgtt taactttaag aaggagatat accatgggtc gtagacgcag gcgtagacgc |
| 61 | aggcgtggtg gcggtccgaa gaaaaagcgt aaagtgggcg gtggcggatc cacgggtgag |
| 121 | aagccgtata aatgtcccga atgtggtaaa agttttagcc gttcgaccca tctgcaacgt |
| 181 | catcaacgca cccataccgg cgaaaaacca tacaaatgtc cggagtgcgg caaatctttc |
| 241 | tcgcgcagcg atcatttgca gcgccatcag agaactcaca ctggcgagaa gccctacaag |
| 301 | tgccccgaat gcgggaagag ctttagtaga tctgatcact tacaaaggca ccagaggacg |
| 361 | cataccggtg agaagccgta taaatgtccc gaatgtggta aaagttttag cacgtcggac |
| 421 | agcctgcaaa cccatcaacg cacccatacc ggcgaaaaac catacaaatg tccggagtgc |
| 481 | ggcaaatctt tctcgcgcag caattcgttg cagcgtcatc agagaactca cactggcgag |
| 541 | aagccctaca agtgccccga atgcgggaag agctttagta gatctgatca tttacaacgc |
| 601 | caccagagga cgcatacggg cgagaaaccg tatggtggcg gtggcagccc taggccgacc |
| 661 | gatgtgagcc tgggcgatga actgcattta gatggcgaag atgtggcgat ggcccatgcg |
| 721 | gatgccctag acgattttga cctggatatg ttaggcgatg gtgacagccc cggtccgggt |
| 781 | tttaccccgc atgatagcgc accgtatggt gcgctagata tggcggattt cgaatttgaa |
| 841 | cagatgttta ccgatgcgct gggtattgat gaatatggcg gtgctagcga ttataaagat |
| 901 | gacgatgaca aataagcggc cgcactcgag caccaccacc accaccactg agatccg |
| LOCUS EPO-1\in\p 957 bp DNA | |
| COMMENT (CGexDoc “EPO-1 in pET21” 1 957 | |
| ORIGDB\|GenBank | |
| (SEQ ID NO: 38) | |
| 1 | taattttgtt taactttaag aaggagatat accatgggtc gtagacgcag gcgtagacgc |
| 61 | aggcgtggtg gcggtccgaa gaaaaagcgt aaagtgggcg gtggcggatc cacgggtgag |
| 121 | aagccgtata aatgtcccga atgtggtaaa agttttagcc gttcgagcaa tctgcaacgt |
| 181 | catcaacgca cccataccgg cgaaaaacca tacaaatgtc cggagtgcgg caaatctttc |
| 241 | tcgcgtagcg atcatttgca gacccatcag agaactcaca ctggcgagaa gccctacaag |
| 301 | tgccccgaat gcgggaagag ctttagtaga tctaatcact tacaacgcca ccagaggacg |
| 361 | cataccggtg agaagccgta taaatgtccc gaatgtggta aaagttttag ccgctcggac |
| 421 | catctgcaaa gacatcaacg cacccatacc ggcgaaaaac catacaaatg tccggagtgc |
| 481 | ggcaaatctt tctcgaccag cgatcacttg cagacgcatc agagaactca cactggcgag |
| 541 | aagccctaca agtgccccga atgcgggaag agctttagta gatctaatag cttacaacgc |
| 601 | caccagagga cgcatacggg cgagaaaccg tatggtggcg gtggcagccc taggccgacc |
| 661 | gatgtgagcc tgggcgatga actgcattta gatggcgaag atgtggcgat ggcccatgcg |
| 721 | gatgccctag acgattttga cctggatatg ttaggcgatg gtgacagccc cggtccgggt |
| 781 | tttaccccgc atgatagcgc accgtatggt gcgctagata tggcggattt cgaatttgaa |
| 841 | cagatgttta ccgatgcgct gggtattgat gaatatggcg gtgctagcga ttataaagat |
| 901 | gacgatgaca aataagcggc cgcactcgag caccaccacc accaccactg agatccg |

//

In one embodiment, the chimeric proteins of the invention bind to promoters of human EPO and activate the transcription of EPO; and in alternative embodiments the EPO activated by the chimeric proteins of the invention (by transcriptional activation of promoters) is a heterologous EPO gene. In one embodiment a human EPO gene such as (SEQ ID NO:39) is used:

| (SEQ ID NO: 39) | |
|---|---|
| 1 | cccggagccg gaccggggcc accgcgcccg ctctgctccg acaccgcgcc ccctggacag |
| 61 | ccgccctctc ctccaggccc gtggggctgg ccctgcaccg ccgagcttcc cgggatgagg |
| 121 | gcccccggtg tggtcacccg gcgcgcccca ggtcgctgag ggaccccggc caggcgcgga |
| 181 | gatgggggtg cacgaatgtc ctgcctggct gtggcttctc ctgtccctgc tgtcgctccc |
| 241 | tctgggcctc ccagtcctgg gcgccccacc acgcctcatc tgtgacagcc gagtcctgga |
| 301 | gaggtacctc ttggaggcca aggaggccga gaatatcacg acgggctgtg ctgaacactg |
| 361 | cagcttgaat gagaatatca ctgtcccaga caccaaagtt aatttctatg cctggaagag |
| 421 | gatggaggtc gggcagcagg ccgtagaagt ctggcagggc ctggccctgc tgtcggaagc |
| 481 | tgtcctgcgg ggccaggccc tgttggtcaa ctcttcccag ccgtgggagc ccctgcagct |
| 541 | gcatgtggat aaagccgtca gtggccttcg cagcctcacc actctgcttc gggctctggg |
| 601 | agcccagaag gaagccatct cccctccaga tgcggcctca gctgctccac tccgaacaat |
| 661 | cactgctgac actttccgca aactcttccg agtctactcc aatttcctcc ggggaaagct |
| 721 | gaagctgtac acaggggagg cctgcaggac aggggacaga tgaccaggtg tgtccacctg |
| 781 | ggcatatcca ccacctccct caccaacatt gcttgtgcca caccctcccc cgccactcct |
| 841 | gaaccccgtc gaggggctct cagctcagcg ccagcctgtc ccatggacac tccagtgcca |
| 901 | gcaatgacat ctcaggggcc agaggaactg tccagagagc aactctgaga tctaaggatg |
| 961 | tcacagggcc aacttgaggg cccagagcag gaagcattca gagagcagct ttaaactcag |
| 1021 | ggacagagcc atgctgggaa gacgcctgag ctcactcggc accctgcaaa atttgatgcc |
| 1081 | aggacacgct ttggaggcga tttacctgtt ttcgcaccta ccatcaggga caggatgacc |
| 1141 | tggagaactt aggtggcaag ctgtgacttc tccaggtctc acgggcatgg gcactccctt |
| 1201 | ggtggcaaga gcccccttga caccggggtg gtgggaacca tgaagacagg atgggggctg |
| 1261 | gcctctggct ctcatggggt ccaagttttg tgtattcttc aacctcattg acaagaactg |
| 1321 | aaaccaccaa aaaaaaaaaa |

In one embodiment, compositions of the invention (e.g., chimeric proteins and/or the nucleic acids that encode them) are administered with EPO, e.g., the human EPO as in SEQ ID NO:40:

(SEQ ID NO: 40)

MGVHECPAWLWLLLSLLSLPLGLPVLGAPPRLICDSRVLERYLL
EAKEAENITTGCAEHCSLNENITVPDTKVNFYAWKRMEVGQQAV
EVWQGLALLSEAVLRGQALLVNSSQPWEPLQLHVDKAVSGLRSL
TTLLRALGAQKEAISPPDAASAAPLRTITADTFRKLFRVYSNFL
RGKLKLYTGEACRTGDR

A number of embodiments of the invention have been described. Nevertheless, it will be understood that various modifications may be made without departing from the spirit and scope of the invention. Accordingly, other embodiments are within the scope of the following claims.

1. A chimeric protein comprising
(a) (1) a plurality of (multiple) DNA-binding domains specific for (that can specifically bind to) a promoter and/or another transcriptional regulatory region of an erythropoietin (EPO) gene or a Erythropoietin Stimulating Protein (NESP) gene;
(2) at least one nuclear localization peptide (NLP) domain;
(3) at least one cell-penetrating peptide (CPP); and,
(4) at least one transcription activation (TA) domain; or (b) the chimeric protein of (a), comprising an EPO-specific zinc finger
DNA-binding domain, or an NESP-specific zinc finger DNA-binding domain.

2. The chimeric protein of claim 1:
(a) wherein the chimeric protein, the EPO- or NESP-specific DNA-binding domain, the NLP domain, the CPP domain and/or the TA domain comprises a recombinant protein, a synthetic protein, a peptidomimetic, a non-natural peptide or a combination thereof;
(b) wherein the EPO or NESP gene or transcriptional regulatory region of an EPO or NESP gene comprises: (a) a mammalian EPO or NESP gene or transcriptional regulatory region or (b) a mouse or a human EPO or NESP gene or transcriptional regulatory region;
(c) wherein the chimeric protein of (b), wherein the transcriptional regulatory region comprises a promoter or an enhancer, an EPO or NESP promoter or EPO or NESP enhancer, or a synthetic promoter;
(d) wherein the chimeric protein comprises multiple copies of the EPO- or NESP-specific DNA-binding domain, the NLP, the CPP and/or the TA domain;
(e) wherein the chimeric protein comprises two, three, four, five, six or more EPO- or NESP-specific DNA-binding domains specific for (that can specifically bind to) a promoter and/or another transcriptional regulatory region of an EPO or NESP gene;
(f) wherein the chimeric protein comprises one, two, three, four, five, six or more nuclear localization peptide (NLP) domains or consensus nuclear localization proteins;
(g) wherein the chimeric protein comprises one, two, three, four, five, six or more cell-penetrating peptides (CPPs) ;
(h) wherein the chimeric protein comprises one, two, three, four, five, six or more TA domains, and/or one or more other functional domains with a histone acetyltransferase (HAT) activity;
(i) wherein the chimeric protein of (h), wherein the at least one TA domain comprises a herpes simplex virus (HSV) VP-16 activation peptide domain or a peptide derived from the C-terminal transcription activation domain of β-catenin (FDTDL) (SEQ ID NO:30);
(j) wherein the at least one zinc finger DNA-binding domain comprises (1) a zinc-finger of the C₂H₂ class; (2) a zinc-finger of the C₄ class; or (3) a zinc-finger of C₆ class;
(k) wherein the at least one zinc finger DNA-binding domain comprises the consensus sequence Cys-X₂₋₄-Cys-X₃-Phe-X₅-Leu-X₂-His-X₃-His (SEQ ID NO:31);
(l) wherein the at least one nuclear localization peptide (NLP) domain comprises
(1) an NLP sequence of a large T antigen of the simian virus 40 (SV-40), or PKKKRKV (SEQ ID NO:29) (SEQ ID NO:2);
(2) a consensus sequence fitting B₄ (SEQ ID NO:32), P(B₃X) (SEQ ID NO:33), PXX(B₃X) (SEQ ID NO:34), B₃(H/P) (SEQ ID NO:35), where B is a basic amino acid, P is proline, H is histidine, X is any amino acid and letters in parentheses can be in any order;
(3) a bipartite NLP comprising two short stretches of basic amino acids separated by a non-conserved sequence; or
(4) a cellular nucleoplasm [[̂]] protein KRPAATKKAGQAKKKK (SEQ ID NO:4);
(m) wherein the at least one cell-penetrating peptide (CPP) comprises
(1) a plurality of polycationic amino acid residues;
(2) a plurality of arginine amino acid residues; or
(3) a TAT protein (Trans-acting Activator of Transcription) of a Human Immunodeficiency Virus (HIV-l) ;
(n) wherein (1) the at least one TA domain is at least approximately 25% hydrophobic and is linked to the at least one zinc finger DNA-binding domain in a manner that does not interfere with the promoter or a transcriptional regulatory binding activity of the zinc finger DNA binding peptide, and the TA domain is both necessary and sufficient to activate transcription of the gene; and/or (2) the TA domain is between about 5 to 25 amino acids in length, or is between about 6 to 20 amino acids in length, or is about 5, 6, 7, 8, 9, 10, 11, 11, 12, 13, 14 or 15 amino acids in length;
(o) wherein the at least one TA domain comprises a herpes simplex virus (I-ISV) VP-16 activation peptide domain or a peptide derived from the C-terminal transcription activation domain of β-catenin (FDTDL) (SEQ ID NO:30);
(p) wherein at least one, or all, of the domains and/or chimeric proteins further comprises, or is attached to, a lipid, a hydrophobic alkane or alkene (olefin) moiety, or a polyethylene glycol (PEG) moiety;
(q) wherein at least one, or all, of the chimeric proteins further comprises, or is attached to, an epitope peptide tag or a detectable composition or moiety;
(r) wherein the detectable composition or moiety comprises a phosphoprotein, a fluorescent molecule, a fluorescent tagged protein, a radiolabel or a radiolabeled protein;
(s) further comprising a small molecule, a hormone or a cytokine that increases or upregulates erythropoiesis or red blood cell production in a mammalian; or
(t) wherein the chimeric protein comprises (a) a formulation for subcutaneous, parenteral, topical, oral or local administration, or for aerosol or transdermal administration, or administration by nebulizer; or (b) the chimeric protein of (a), wherein the topical formulation comprises an ointment, a cream, a powder, an emulsion, a gel, a glycerogelatin, a paste, a plaster, a sprayable composition or a lotion.

3-21. (canceled)

22. A composition comprising:
(a) a plurality of chimeric proteins of claim 1;
(b) the composition of (a), further comprising a small molecule, a hormone or a cytokine that increases or upregulates erythropoiesis or red blood cell production in a mammalian;
(c) the composition of (a), further comprising a synthetic or recombinant erythropoietin;
(d) the composition of (a), further comprising or formulated as a liquid, gel, hydro gel, powder or aqueous formulation, or a vesicle, liposome, nanoparticle or nanolipid particle (NLP);
(e) the composition of (a), further comprising or formulated as or is contained in an isolated or cultured cell, or a mammalian cell, or a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell;
(f) the composition of any of (a) to (d), further comprising or formulated as or is contained in a pharmaceutical or sterile formulation; or
(g) the composition of any of (a) to (d), further comprising or formulated as or is contained in a product of manufacture.

23-31. (canceled)

32. A recombinant or synthetic nucleic acid comprising:
(a) a nucleic acid sequence encoding the chimeric protein of claim 1;
(b) the recombinant or synthetic nucleic acid of (a) contained in a vector, plasmid, recombinant virus or expression vehicle, and optionally the nucleic acid is operatively linked to a constitutive promoter or an inducible promoter or a promoter only active in a hematopoietic cell; or
(c) the recombinant or synthetic nucleic acid of (a) contained in an isolated or cultured cell, or a mammalian cell, or a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell.

33-37. (canceled)

38. A method for ameliorating or preventing an anemia, and/or stimulating erythropoiesis and/or erythropoietin (EPO) synthesis, in an individual comprising:
(1) (a) providing: the pharmaceutical or sterile formulation of claim 30; and
(b) administering an effective amount of (a) to an individual in need thereof;
(2) the method of (1), wherein the individual in need thereof is a mammalian or a human;
(3) the method of (1), wherein the anemia ameliorated or prevented is caused by a genetic disorder, an infection, a dietary disorder or deficiency, a pollutant, a pesticide, herbicide or insecticide, a poison, a venom, a toxin, a biological agent, a drug, a cancer or a cancer therapeutic or cancer therapy;
(4) the method of (1), wherein the anemia ameliorated or prevented is a microcytic, normocytic or macrocytic form of anemia, or the anemia ameliorated or prevented is: a drug-induced anemia; caused by an infection; caused by an iron deficiency; caused by rhesus disease (hemolytic disease of newborn); caused by sickle-cell disease, thalassemia or Plummer-Vinson syndrome (PVS, also called Paterson-Brown-Kelly syndrome or sideropenic dysphagia); a sideroblastic anemia-congenital or acquired; caused by Gaucher's disease; caused by a vitamin deficiency; caused by autoimmune hemolytic anemia (AIHA); caused by a cancer; or, caused by heavy metal poisoning or pyridoxine deficiency;
(5) the method of (4), wherein the vitamin deficiency is a folate or B 12 deficiency (pernicious anemia o_{f} Addison's anemia);
(6) the method of (4), wherein the drug-induced anemia is caused by methyldopa or fludarabin);
(7) the method of (4), wherein the AIHA is caused by Systemic lupus erythematosus, a drug, Evans syndrome, chronic lymphocytic leukemia or is idiopathic);
(8) the method of (4), wherein the cancer is chronic lymphocytic leukemia, small cell lymphoma (or small lymphocytic lymphoma) or a non-Hodgkin's lymphoma; or the anemia is caused by myelophythisis secondary to an acute megakaryoblastic leukemia, a lymphoma, a myeloma or a carcinoma metastatic to bone marrow);
(9) the method of (4), wherein the infection is an EBV infectious mononucleosis, a Babesiosis infection, or equine infectious anemia);
(10) the method of (4), wherein the cancer therapeutic or cancer therapy is radiotherapy, hormone therapy or chemotherapy); or
(11) the method of (4), wherein the heavy metal poisoning is lead poisoning, mercury poisoning (hydrargaria), copper poisoning, nickel poisoning, manganese poisoning (manganism) or cadmium poisoning.

39-80. (canceled)
